SEQUENCE LISTING (93 sequences)

SEQ ID NO: 1 - protein, 119 aa, Homo sapians
QVQLQESGPGLVKPSQTLSLTCTVSGGSISSGGYYWSWIRQHPGKGLEWIGEINHSGSTNYNPSLKSRVTISVDTSKNQFSLKLSSVTAADTAVYYCARGRDGYDFDPWGQGTLVTVSS

SEQ ID NO: 2 - protein, 111 aa, Homo sapians
LPVLTQPASVSGSPGQSITISCTGTSSDVGGYNYVSWYQQHPGKAPKLMIYDVSDRPSGVSTRFSGSKSGNTASLTISGLQAEDEADYYCSSYRSSSALVVFGGGTKLTVL

SEQ ID NO: 3 - protein, 119 aa, Homo sapians
QVQLQESGPGLVKPSGTLSLTCAVSGGSISRSNWWSWVRQPPGKGLEWIGEVYHSGSTNYNPSLKSRVTISVDKSKNQFSLKVNSVTAADTAVYYCARDSDGGYYFDYWGQGTLVTVSS

SEQ ID NO: 4 - protein, 111 aa, Homo sapians
LPVLTQPASVSGSPGQSITISCTGTSSDVGGYKYVSWYQQHPGKAPKLLIYDVTDRPSGVSNRFSGSQSGNTASLTISGLQTEDEADYYCSSYTDNGALVVFGGGTKLTVL

SEQ ID NO: 5 - protein, 127 aa, Homo sapians
QITLKESGAEVKKPGSSVKVSCKASGGTFSSYGISWVRQAPGQGLEWMGGIIPMFGTANYAQKFQGRVTITADESTSTAYMELSSLRSEDTAVYYCARDEVAPDYYGSGPSYGMDVWGQGTMVTVSS

SEQ ID NO: 6 - protein, 110 aa, Homo sapians
SYELMQPASVSGSPGQSITISCTGTSSDVGGYDHVSWYQQHPGKAPKLMIYAVRNRPSGVPDRFSGSKSGNTASLTISGLQAEDEADYYCSSYTSSLTYVFGTGTKVTVL

SEQ ID NO: 7 - protein, 118 aa, Homo sapians
QVQLVESGAEVKKPGASVKVSCKASGYTFTGYYMHWVRQAPGQGLEWMGWINPNSGNTGYAQKFQGRVTMTRNTSISTAYMELSSLRSEDTAVYYCARRGTTVSFDYWGQGTTVTVSS

SEQ ID NO: 8 - protein, 110 aa, Homo sapians
QAVLTQPPSVSGSPGQSITISCTGTSSDVGTFNLVSWYQQHPGKAPKLIIYEVSKRPSDVSPRYSGSKSGTTASLTISVLQTEDEADYYCCSYTTSSSYVFGIGTKVTVL

SEQ ID NO: 9 - protein, 117 aa, Homo sapians
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRDGYDFDPWGQGTLVTVSS

SEQ ID NO: 10 - protein, 111 aa, Homo sapians
QSVLTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVVFGGGTQLTVL

SEQ ID NO: 11 - protein, 123 aa, Homo sapians
QVQLVQSGAEVKKPGASVKVSCKTSGYTFSGDYMHWVRQAPGQGLEWMGWINPNSGGTNYAQKFQGRVTMTRDTSISTAYMELSRLRSDDTAVYYCAREPGRDYYYYDGMDVWGQGTTVTVSS

SEQ ID NO: 12 - protein, 111 aa, Homo sapians
QSVVTQPPSVSGAPGQRVTISCTGSSSNIGAGYDVHWYQQLPGTVPKLLIYGNSNRPSGVPDRFSGSKSGTSASLAITGLQAEDEADYYCQSYDSSLSAYVFGTGTKVTVL

SEQ ID NO: 13 - protein, 120 aa, Homo sapians
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGRVYSNYYMDVWGKGTTVTVSS

SEQ ID NO: 14 - protein, 111 aa, Homo sapians
QAVLTQPASVSGSPGQSITISCTGTRSDVGGYNYVSWYQQHPGKAPKLLVYDVSNRPSGVSNRFSGSQSGNTASLTISGLQTEDEADYYCSSYTDNSALVVFGGGTKVTVL

SEQ ID NO: 15 - protein, 119 aa, Homo sapians
QVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARSAYGDYFLDYWGQGTLVTVSS

SEQ ID NO: 16 - protein, 111 aa, Homo sapians
QSVLTQPASVSGSPGQSITISCTGTSSDVGGYNYVSWYQQHPGKAPKLLIYDVDSRPSGVSNRFSGSKSGNTASLTISGLQAEDEADYYCSSFTSSSTLVVFGGGTKVTVL

SEQ ID NO: 17 - protein, 120 aa, Homo sapians
EVQLLESGGGLVQPGGSLRLSCAASGFTFSSYEMNWVRQAPGKGLEWVSYISSSGSTIYYADSVKGRFTISRDNAKNSLYLQMNSLRAEDTAVYYCARDGAATGDQIDYWGQGTLVTVSS

SEQ ID NO: 18 - protein, 107 aa, Homo sapians
AIRMTQSPAFMSATPGDKVNISYKASQDVDDDMTWCQEKPGEAAIFIFQEAATLVPGIPPRLSGSGNGTDFTLTINNMESEDAAYYFCLQQDNFPLTFGQGTKVDIK

SEQ ID NO: 19 - protein, 117 aa, Homo sapians
EVQLVQSGAEVKKPGASVKVSCKASGYTFSSYYMHWVRQAPGQGLEWMGWINPNSGNTGYAQKFQGRVTMTRNTSISTAYMELSSLRSEDTAVYYCARRGTTVSFDYWGQGTLVTVS

SEQ ID NO: 20 - protein, 110 aa, Homo sapians
QLVLTQSPSVSVAPGQRVTISCSGSNSNIGNNYVSWYHHLPGTAPKLLIYDNNKRPSGIPDRFSGSKSGTSATLGITGLQPGDEAHYYCGTWDSTLSAGVFGGGTKLTVL

SEQ ID NO: 21 - protein, 118 aa, Homo sapians
EVQLVQSGAEVKKPGASVKVSCKASGYTFYSYYMHWVRQAPGQGLEWMGWINPNSGNTGYAQKFQGRVTMTRNTSISTAYMELSSLRSEDTAVYYCARRGTTVSFDTWGQGTLVTVSS

SEQ ID NO: 22 - protein, 110 aa, Homo sapians
QLVLTQSPSVSVAPGQRVTISCSGSNSNIGNNYVSWYHHLPGTAPKLLIYDNNKRPSGIPDRFSGSKSGTSATLGITGLQPGDEAHYYCGTWDSTLSAWVFGGGTKLTVL

SEQ ID NO: 23 - protein, 110 aa, Homo sapians
QLVLTQSPSVSVAPGQRVTISCSGSNSNIGNNYVSWYHHLPGTAPKLLIYDNNKRPSGIPDRFSGSKSGTSATLGITGLQPGDEAHYYCGTWDSTLSAWLFGGGTKLTVL

SEQ ID NO: 24 - protein, 118 aa, Homo sapians
EVQLVQSGAEVKKPGASVKVSCKASGYTFYSEYMHWVRQAPGQGLEWMGWINPNSGNTGYAQKFQGRVTMTRNTSISTAYMELSSLRSEDTAVYYCARRGTTVSFDTWGQGTLVTVSS

SEQ ID NO: 25 - protein, 118 aa, Homo sapians
EVQLVQSGAEVKKPGASVKVSCKASGYTFYSEYMHWVRQAPGQGLEWMGWINPNSGNTGVAPKFQGRVTMTRNTSISTAYMELSSLRSEDTAVYYCARRGTTVSFDTWGQGTLVTVSS

SEQ ID NO: 26 - protein, 110 aa, Homo sapians
QLVLTQSPSVSVAPGQRVTISCSGSNSNIGNNYVSWYHHLPGTAPKLLIYDNNKRPSGIPDRFSGSKSGTSATLGITGLQPGDEAHYYCGTWDSTLSAWAFGGGTKLTVL

SEQ ID NO: 27 - protein, 118 aa, Homo sapians; identical to SEQ ID NO: 24

SEQ ID NO: 28 - protein, 110 aa, Homo sapians
QLVLTQSPSVSVAPGQRVTISCSGSNSFTDNTYVSWYHHLPGTAPKLLIYDTNKRPSGIPDRFSGSKSGTSATLGITGLQPGDEAHYYCGTWDSTLSAWVFGGGTKLTVL

SEQ ID NO: 29 - protein, 118 aa, Homo sapians
EVQLVQSGAEVKKPGASVKVSCKASGYTFYSEYMHWVRQAPGQGLEWMGWINPNSGNTGLAPKFQGRVTMTRNTSISTAYMELSSLRSEDTAVYYCARRGTTVSFDTWGQGTLVTVSS

SEQ ID NO: 30 - protein, 110 aa, Homo sapians
QLVLTQSPSVSVAPGQRVTISCSGSNSNIGNNYVSWYHHLPGTAPKLLIYDNNKRQSGIPDRFSGSKSGTSATLGITGLQPGDEAHYYCGTWDSTLSAWVFGGGTKLTVL

SEQ ID NO: 31 - protein, 118 aa, Homo sapians
EVQLVQSGAEVKKPGASVKVSCKASGYTFYSYYMHWVRQAPGQGLEWMGWINPNSGNTGVAQKFQGRVTMTRNTSISTAYMELSSLRSEDTAVYYCARRGTTVSFDTWGQGTLVTVSS

SEQ ID NO: 32 - protein, 118 aa, Homo sapians
EVQLVQSGAEVKKPGASVKVSCKASGYTFYSEYMHWVRQAPGQGLEWMGWINPNSGNTGLAQKFQGRVTMTRNTSISTAYMELSSLRSEDTAVYYCARRGTTVSFDTWGQGTLVTVSS

SEQ ID NO: 33 - protein, 118 aa, Homo sapians
EVQLVQSGAEVKKPGASVKVSCKASGYTFYSEYMHWVRQAPGQGLEWMGWINPNSGNTGYAPKFQGRVTMTRNTSISTAYMELSSLRSEDTAVYYCARRGTTVSFDTWGQGTLVTVSS

SEQ ID NO: 34 - protein, 118 aa, Homo sapians
EVQLVQSGAEVKKPGASVKVSCKASGYTFYSEYMHWVRQAPGQGLEWMGWINPNSGNTGVAQKFQGRVTMTRNTSISTAYMELSSLRSEDTAVYYCARRGTTVSFDTWGQGTLVTVSS

SEQ ID NO: 35 - protein, 110 aa, Homo sapians
QLVLTQSPSVSVAPGQRVTISCSGSNSFSSNNYVSWYHHLPGTAPKLLIYDNNKRPSGIPDRFSGSKSGTSATLGITGLQPGDEAHYYCGTWDSTLSAWVFGGGTKLTVL

SEQ ID NO: 36 - protein, 118 aa, Homo sapians
EVQLVQSGAEVKKPGASVKVSCKASGYTFYSYYMHWVRQAPGQGLEWMGWINPNSGNTGLAQKFQGRVTMTRNTSISTAYMELSSLRSEDTAVYYCARRGTTVSFDTWGQGTLVTVSS

SEQ ID NO: 37 - protein, 118 aa, Homo sapians
EVQLVQSGAEVKKPGASVKVSCKASGYTFYSYYMHWVRQAPGQGLEWMGWINPNSGNTGLAPKFQGRVTMTRNTSISTAYMELSSLRSEDTAVYYCARRGTTVSFDTWGQGTLVTVSS

SEQ ID NO: 38 - protein, 110 aa, Homo sapians
QLVLTQSPSVSVAPGQRVTISCSGSNSNIGNNYVSWYHHLPGTAPKLLIYDTNKRPSGIPDRFSGSKSGTSATLGITGLQPGDEAHYYCGTWDSTLSAWAFGGGTKLTVL

SEQ ID NO: 39 - protein, 110 aa, Homo sapians
QLVLTQSPSVSVAPGQRVTISCSASNSNIGNNYVSWYHHLPGTAPKLLIYDNNKRPSGIPDRFSGSKSGTSATLGITGLQPGDEAHYYCGTWDSTLSAWVFGGGTKLTVL

SEQ ID NO: 40 - protein, 123 aa, Homo sapians
QVQLVQSGAEVKKPGASVKVSCKASGYTFSGDYMHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 41 - protein, 111 aa, Homo sapians
QSVVTQPPSVSGAPGQRVTISCLGSASNIGAGHDVHWYQQLPGTAPKLLIYGNSNRPSGVPDRFSGSKSGTSASLAITGLQAEDEADYYCQSYSSSLSAYVFGTGTKVTVL

SEQ ID NO: 42 - protein, 123 aa, Homo sapians
QVQLVQSGAEVKKPGASVKVSCKASGFTFSGDYIHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 43 - protein, 111 aa, Homo sapians
QSVVTQPPSVSGAPGQRVTISCLGSSSNIGAGHDVHWYQQLPGTAPKLLIYGNSNRISGVPDRFSGSKSGTSASLAITGLQAEDEADYYCQSYSSSLSAYLFGTGTKVTVL

SEQ ID NO: 44 - protein, 123 aa, Homo sapians
QVQLVQSGAEVKKPGASVKVSCKASGYTFSGDYLHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 45 - protein, 123 aa, Homo sapians
QVQLVQSGAEVKKPGASVKVSCKASGYTFSGDYIHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 46 - protein, 111 aa, Homo sapians
QSVVTQPPSVSGAPGQRVTISCIGSSSNIGAGHDVHWYQQLPGTAPKLLIYGNSNRPSGVPDRFSGSKSGTSASLAITGLQAEDEADYYCQSYSSSLSAYVFGTGTKVTVL

SEQ ID NO: 47 - protein, 123 aa, Homo sapians
QVQLVQSGAEVKKPGASVKVSCKASGYTFSGDYLHWVRQAPGQGLEWMGWINPNTGGTNYAQKFQGRVTMTRDTSISTAYMELSRLRSDDTAVYYCAREPARDYYYYDGLDVWGQGTTVTVSS

SEQ ID NO: 48 - protein, 111 aa, Homo sapians
QSVVTQPPSVSGAPGQRVTISCIGSSSNIGAGYDVHWYQQLPGTAPKLLIYGNSNLPSGVPDRFSGSKSGTSASLAITGLQAEDEADYYCQSYESSLSAYVFGTGTKVTVL

SEQ ID NO: 49 - protein, 123 aa, Homo sapians
QVQLVQSGAEVKKPGASVKVSCKASGYTFSGDYMHWVRQAPGQGLEWMGWINPNSGGTNYAQKFQGRVTMTRDTSISTAYMELSRLRSDDTAVYYCAREPGRDYYYYDGLDVWGQGTTVTVSS

SEQ ID NO: 50 - protein, 111 aa, Homo sapians
QSVVTQPPSVSGAPGQRVTISCIGSASNIGAGHDVHWYQQLPGTAPKLLIYGNSNRPSGVPDRFSGSKSGTSASLAITGLQAEDEADYYCQSYSSSLSAYVFGTGTKVTVL

SEQ ID NO: 51 - protein, 123 aa, Homo sapians
QVQLVQSGAEVKKPGASVKVSCKASGYTFSGDYMHWVRQAPGQGLEWMGWINPNTGGTNYAQKFQGRVTMTRDTSISTAYMELSRLRSDDTAVYYCAREPARDYYYYDGLDVWGQGTTVTVSS

SEQ ID NO: 52 - protein, 111 aa, Homo sapians
QSVVTQPPSVSGAPGQRVTISCLGSASNIGAGYDVHWYQQLPGTAPKLLIYGNSNRPSGVPDRFSGSKSGTSASLAITGLQAEDEADYYCQSYSSSLSAYVFGTGTKVTVL

SEQ ID NO: 53 - protein, 123 aa, Homo sapians
QVQLVQSGAEVKKPGASVKVSCKASGFTFSGDYMHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 54 - protein, 111 aa, Homo sapians
QSVVTQPPSVSGAPGQRVTISCLGSSSNIGAGHDVHWYQQLPGTAPKLLIYGNSNLPSGVPDRFSGSKSGTSASLAITGLQAEDEADYYCQSYSSSLSAVLFGTGTKVTVL

SEQ ID NO: 55 - protein, 111 aa, Homo sapians
QSVVTQPPSVSGAPGQRVTISCIGSSSNIGAGYDVHWYQQLPGTAPKLLIYGNSNRISGVPDRFSGSKSGTSASLAITGLQAEDEADYYCQSYSSSLSAVLFGTGTKVTVL

SEQ ID NO: 56 - protein, 123 aa, Homo sapians
QVQLVQSGAEVKKPGASVKVSCKASGSTFSGDYIHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 57 - protein, 111 aa, Homo sapians
QSVVTQPPSVSGAPGQRVTISCLGSASNIGAGHDVHWYQQLPGTAPKLLIYGNSNRPSGVPDRFSGSKSGTSASLAITGLQAEDEADYYCQSYSSSLSAVLFGTGTKVTVL

SEQ ID NO: 58 - protein, 123 aa, Homo sapians
QVQLVQSGAEVKKPGASVKVSCKASGFTFSGDYIHWVRQAPGQGLEWMGWINPNSGGTNYAQKFQGRVTMTRDTSISTAYMELSRLRSDDTAVYYCAREPGRDYYYYDGLDVWGQGTTVTVSS

SEQ ID NO: 59 - protein, 123 aa, Homo sapians
QVQLVQSGAEVKKPGASVKVSCKASGSTFSGDYLHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 60 - protein, 111 aa, Homo sapians
QSVVTQPPSVSGAPGQRVTISCLGSASNIGAGYDVHWYQQLPGTAPKLLIYGNSNRPSGVPDRFSGSKSGTSASLAITGLQAEDEADYYCQSYSSSLSAVLFGTGTKVTVL

SEQ ID NO: 61 - protein, 123 aa, Homo sapians
QVQLVQSGAEVKKPGASVKVSCKASGYTFSGDYLHWVRQAPGQGLEWMGWINPNSGGTNYAQKFQGRVTMTRDTSISTAYMELSRLRSDDTAVYYCAREPARDYYYYDGMDVWGQGTTVTVSS

SEQ ID NO: 62 - protein, 111 aa, Homo sapians
QSVVTQPPSVSGAPGQRVTISCLGSASNIGAGHDVHWYQQLPGTAPKLLIYGNSNRISGVPDRFSGSKSGTSASLAITGLQAEDEADYYCQSYSSSLSAVLFGTGTKVTVL

SEQ ID NO: 63 - protein, 123 aa, Homo sapians
QVQLVQSGAEVKKPGASVKVSCKASGFTFSGDYLHWVRQAPGQGLEWMGWINPNSGGTNYAQKFQGRVTMTRDTSISTAYMELSRLRSDDTAVYYCAREPARDYYYYYGLDVWGQGTTVTVSS

SEQ ID NO: 64 - protein, 111 aa, Homo sapians
QSVVTQPPSVSGAPGQRVTISCLGSSSNIGAGHDVHWYQQLPGTAPKLLIYGNSNRISGVPDRFSGSKSGTSASLAITGLQAEDEADYYCQSYSSSLSAYVFGTGTKVTVL

SEQ ID NO: 65 - protein, 123 aa, Homo sapians
QVQLVQSGAEVKKPGASVKVSCKASGFTFSGDYLHWVRQAPGQGLEWMGWINPNTGGTNYAQKFQGRVTMTRDTSISTAYMELSRLKSDDTAVYYCAREPARDYYYYDGLDVWGQGTTVTVSS

SEQ ID NO: 66 - protein, 111 aa, Homo sapians
QSVVTQPPSVSGAPGQRVTISCLGSSSNIGAGHDVHWYQQLPGTAPKLLIYGNSNLPSGVPDRFSGSKSGTSASLAITGLQAEDEADYYCQSYSSSLSAYVFGTGTKVTVL

SEQ ID NO: 67 - protein, 111 aa, Homo sapians
QSVVTQPPSVSGAPGQRVTISCLGSASNIGAGHDVHWYQQLPGTAPKLLIYGNSNRPSGVPDRFSGSKSGTSASLAITGLQAEDEADYYCQSYSSSLSAVVFGTGTKVTVL

SEQ ID NO: 68 - protein, 111 aa, Homo sapians
QSVVTQPPSVSGAPGQRVTISCIGSASNIGAGHDVHWYQQLPGTAPKLLIYGNSNRISGVPDRFSGSKSGTSASLAITGLQAEDEADYYCQSYSSSLSAVLFGTGTKVTVL

SEQ ID NO: 69 - protein, 123 aa, Homo sapians
QVQLVQSGAEVKKPGASVKVSCKASGFTFSGDYLHWVRQAPGQGLEWMGWINPNSGGTNYAQKFQGRVTMTRDTSISTAYMELSRLRSDDTAVYYCAREPARDYYYYDGLDVWGQGTTVTVSS

SEQ ID NO: 70 - protein, 111 aa, Homo sapians
QSVVTQPPSVSGAPGQRVTISCLGSSSNIGAGHDVHWYQQLPGTAPKLLIYGNSNRPSGVPDRFSGSKSGTSASLAITGLQAEDEADYYCQSYSSSLSAYLFGTGTKVTVL

SEQ ID NO: 71 - protein, 123 aa, Homo sapians
QVQLVQSGAEVKKPGASVKVSCKASGYTFSGDYMHWVRQAPGQGLEWMGWINPNSGGTNYAQKFQGRVTMTRDTSISTAYMELSRLRSDDTAVYYCAREPGRDYYYYDGMDVWGQGTTVTVSS

SEQ ID NO: 72 - protein, 111 aa, Homo sapians
QSVVTQPPSVSGAPGQRVTISCLGSSSNIGAGYDVHWYQQLPGTAPKLLIYGNSNRPSGVPDRFSGSKSGTSASLAITGLQAEDEADYYCQSYSSSLSAYVFGTGTKVTVL

SEQ ID NO: 73 - protein, 111 aa, Homo sapians
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYSSSLSAYVFGTGTKVTVL

SEQ ID NO: 74 - protein, 123 aa, Homo sapians
QVQLVQSGAEVKKPGASVKVSCKASGSTFSGDYLHWVRQAPGQGLEWMGWINPNSGGTNYAQKFQGRVTMTRDTSISTAYMELSRLRSDDTAVYYCAREPGRDYYYYDGLDVWGQGTTVTVSS

SEQ ID NO: 75 - protein, 123 aa, Homo sapians
QVQLVQSGAEVKKPGASVKVSCKASGFTFSGDYLHWVRQAPGQGLEWMGWINPNSGGTNYAQKFQGRVTMTRDTSISTAYMELSRLRSDDTAVYYCAREPARDYYYYDGMDVWGQGTTVTVSS

SEQ ID NO: 76 - protein, 123 aa, Homo sapians
QVQLVQSGAEVKKPGASVKVSCKASGYTFSGDYLHWVRQAPGQGLEWMGWINPNSGGTNYAQKFQGRVTMTRDTSISTAYMELSRLRSDDTAVYYCAREPGRDYYYYDGLDVWGQGTTVTVSS

SEQ ID NO: 77 - protein, 110 aa, Homo sapians
QLVLTQSPSVSVAPGQRVTISCSGSNSFIGNNYVSWYHHLPGTAPKLLIYDNNKRPSGIPDRFSGSKSGTSATLGITGLQPGDEAHYYCGTWDSTLSAWVFGGGTKLTVL

SEQ ID NO: 78 - protein, 110 aa, Homo sapians
QLVLTQSPSVSVAPGQRVTISCSGSNSNIGNTYVSWYHHLPGTAPKLLIYDNNKRPSGIPDRFSGSKSGTSATLGITGLQPGDEAHYYCGTWDSTLSAWVFGGGTKLTVL

SEQ ID NO: 79 - protein, 118 aa, Homo sapians
EVQLVQSGAEVKKPGASVKVSCKASGYTFSDYYMHWVRQAPGQGLEWMGWINPNSGNTGYAQKFQGRVTMTRNTSISTAYMELSSLRSEDTAVYYCARRATTVSFDYWGQGTLVTVSS

SEQ ID NO: 80 - protein, 119 aa, Homo sapians
QVQLQESGPGLVKPSQTLSLTCTVSGGSISSGGYYWSWIRQHPGKGLEWIGESTHSGSTNYNPSLKSRVTISVDTSKNQFSLKLSSVTAADTAVYYCARGRDGYDFDAWGQGTLVTVSS

SEQ ID NO: 81 - protein, 111 aa, Homo sapians
LPVLTQPASVSGSPGQSITISCTGTSFDVGGYNYVSWYQQHPGKAPKLMIYDVSDRPSGVSTRFSGSKSGNTASLTISGLQAEDEADYYCSSFRSSSALVVFGGGTKLTVL

SEQ ID NO: 82 - protein, 119 aa, Homo sapians
QVQLQESGPGLVKPSQTLSLTCTVSGGSISSGGYYWSWIRQHPGKGLEWIGESSHSGSTNYNPSLKSRVTISVDTSKNQFSLKLSSVTAADTAVYYCARGRDGYYFDAWGQGTLVTVSS

SEQ ID NO: 83 - protein, 111 aa, Homo sapians
LPVLTQPASVSGSPGQSITISCTGTSSDVGGYPYVSWYQQHPGKAPKLMIYVVSDRPSGVSTRFSGSKSGNTASLTISGLQAEDEADYYCSSYRSSSALVVFGGGTQLTVL

SEQ ID NO: 84 - protein, 119 aa, Homo sapians
QVQLQESGPGLVKPSQTLSLTCTVSGGSISSGGYYWSWIRQHPGKGLEWIGEITHSGSTNYNPSLKSRVTISVDTSKNQFSLKLSSVTAADTAVYYCARGRDGYDIDAWGQGTLVTVSS

SEQ ID NO: 85 - protein, 111 aa, Homo sapians
LPVLTQPASVSGSPGQSITISCTGTSWDVGGYPYVSWYQQHPGKAPKLMIYDVSDRPSGVSTRFSGSKSGNTASLTISGLQAEDEADYYCSSYRSVSALVVFGGGTKLTVL

SEQ ID NO: 86 - protein, 119 aa, Homo sapians
QVQLQESGPGLVKPSQTLSLTCTVSGGSISSGGYYWSWIRQHPGKGLEWIGEISHSGSTNYNPSLESRVTISVDTSKNQFSLKLSSVTAADTAVYYCARGRDGYDLDRWGQGTLVTVSS

SEQ ID NO: 87 - protein, 111 aa, Homo sapians
LPVLTQPASVSGSPGQSITISCTGTSSDVGGYPYVSWYQQHPGKAPKLMIYRVSDRPSGVSTRFSGSKSGNTASLTISGLQAEDEADYYCSSYRSSAALVVFGGGTKLTVL

SEQ ID NO: 88 - protein, 119 aa, Homo sapians
QVQLQESGPGLVKPSQTLSLTCTVSGGSISSGGYYWSWIRQHPGKGLEWIGEISHSGSTNYNPSLKSRVTISVDTSKNQFSLKLSSVTAADTAVYYCARGRDGYYLDQWGQGTLVTVSS

SEQ ID NO: 89 - protein, 111 aa, Homo sapians
LPVLTQPASVSGSPGQSITISCTGTSSDVGGYNYVSWYQQHPGKAPKLMIYNVSDRPSGVSTRFSGSKSGNTASLTISGLQAEDEADYYCSSFRSSSALVVFGGGTKLTVL

SEQ ID NO: 90 - protein, 119 aa, Homo sapians
QVQLQESGPGLVKPSQTLSLTCTVSGGSISSGGYYWSWIRQHPGKGLEWIGESTHSGSTNYNPSLESRVTISVDTSKNQFSLKLSSVTAADTAVYYCARGRDSYDFDAWGQGTLVTVSS

SEQ ID NO: 91 - protein, 111 aa, Homo sapians
LPVLTQPASVSGSPGQSITISCTGTSFDVGGYNYVSWYQQHPGKAPKLMIYDVSDRPSGVSTRFSGSKSGNTASLTISGLQAEDEADYYCSSFRSSAALVVFGGGTKLTVL

SEQ ID NO: 92 - protein, 119 aa, Homo sapians
QVQLQESGPGLVKPSQTLSLTCTVSGGSISSGGYYWSWIRQHPGKGLEWIGESTHSGSTNYNPSLDSRVTISVDTSKNQFSLKLSSVTAADTAVYYCARGRDGYYLDQWGQGTLVTVSS

SEQ ID NO: 93 - protein, 111 aa, Homo sapians
LPVLTQPASVSGSPGQSITISCTGTSFDVGGYPYVSWYQQHPGKAPKLMIYDVSDRPSGVSTRFSGSKSGNTASLTISGLQAEDEADYYCSSFRSSAALVVFGGGTKLTVL

(19) United States
(12) Patent Application Publication
(10) Pub. No.: US 2018/0280531 A1
(43) Pub. Date: Oct. 4, 2018

(54) C-MET ANTIBODY DRUG CONJUGATE

(71) Applicant: Sorrento Therapeutics, Inc., San Diego, CA (US)

(72) Inventors: Tong Zhu, San Diego, CA (US); Gang Chen, San Diego, CA (US); Yanwen Fu, San Diego, CA (US); Edwige Gros, San Diego, CA (US)

(21) Appl. No.: 15/533,360

(22) PCT Filed: Dec. 8, 2015
(86) PCT No.: PCT/US15/64571

§ 371 Date: Jun. 5, 2017

Related U.S. Application Data
(60) Provisional application No. 62/089,203, filed on Dec. 8, 2014.

(51) Int. Cl. A61K 47/68; C07K 16/40; C07K 16/28; A61K 47/64

(52) U.S. Cl. CPC A61K 47/6849; C07K 16/40; A61K 2039/505; C07K 16/2863; A61K 47/64; A61K 47/6809

(57) ABSTRACT

There is disclosed an antibody drug conjugate (ADC) having an IgG antibody that binds to a c-Met target conjugated at both Cys sites in the hinge region of an IgG antibody. There is further disclosed a method for treating a breast cancer comprising providing an effective amount of a c-Met ADC.

### TECHNICAL FIELD

The present disclosure provides an antibody drug conjugate (ADC) having an IgG antibody that binds to a c-Met target conjugated at both Cys sites in the hinge region of an IgG antibody. The present disclosure further provides a method for treating a breast cancer comprising providing an effective amount of a c-Met ADC.

### BACKGROUND

HGF is a mesenchyme-derived pleiotrophic factor with mitogenic, motogenic and morphogenic activities on a number of different cell types. HGF effects are mediated through a specific tyrosine kinase, c-Met, and aberrant HGF and c-Met expression are frequently observed in a variety of tumors. (Maulik et al., Cytokine & Growth Factor Reviews (2002), 13:41-59; Danilkovitch-Miagkova & Zbar, J. Clin. Invest. (2002), 109(7):863-867). Regulation of the HGF/c-Met signaling pathway is implicated in tumor progression and metastasis. (Trusolino & Comoglio, Nature Rev. (2002), 2:289-300).

HGF binds the extracellular domain of the Met receptor tyrosine kinase (RTK) and regulates diverse biological processes such as cell scattering, proliferation, and survival. HGF-Met signaling is essential for normal embryonic development especially in migration of muscle progenitor cells and development of the liver and nervous system (Bladt et al., Nature 376, 768-771. 1995; Hamanoue et al. J. Neurosci. Res. 43, 554-564. 1996; Schmidt et al., Proc. Natl. Acad. Sci. USA 94, 11445-11450, 1995; Uehara et al., Nature 373, 702-705, 1995). Developmental phenotypes of Met and HGF knockout mice are very similar suggesting that HGF is the cognate ligand for the Met receptor (Schmidt et al., Proc. Natl. Acad. Sci. USA 94, 11445-11450, 1995; Uehara et al., Nature 373, 702-705, 1995). HGF-Met also plays a role in liver regeneration, angiogenesis, and wound healing (Bussolino et al., J. Cell Biol. 119, 629-641 1992; Nusrat et al., J. Clin. Invest. 93, 2056-2065 1994). The precursor Met receptor undergoes proteolytic cleavage into an extracellular subunit and membrane spanning subunit linked by disulfide bonds (Tempest et al., Br. J. Cancer 58, 3-7 1988). The subunit contains the cytoplasmic kinase domain and harbors a multi-substrate docking site at the C-terminus where adapter proteins bind and initiate signaling. Upon HGF binding, activation of Met leads to tyrosine phosphorylation and downstream signaling through Gab1 and Grb2/Sos mediated PI3-kinase and Ras/MAPK activation respectively, which drives cell motility and proliferation (Furge et al., Oncogene 19, 5582-5589 2000; Hartmann et al., J. Biol. Chem. 269, 21936-21939 1994; Ponzetto et al., Cell 87, 531-542 1996; and Royal and Park, J. Biol. Chem. 270, 27780-27787 1995).

Met overexpression or gene-amplification has been observed in a variety of human cancers. For example, Met protein is overexpressed at least 5-fold in colorectal cancers and reported to be gene-amplified in liver metastasis (Di Renzo et al., Clin. Cancer Res. 1, 147-154, 1995; Liu et al., Oncogene 7, 181-185 1992). Met protein is also reported to be overexpressed in oral squamous cell carcinoma, hepatocellular carcinoma, renal cell carcinoma, breast carcinoma, and lung carcinoma (Jin et al., Cancer 79, 749-760 1997; Morello et al., J. Cell Physiol. 189, 285-290 2001; Natali et al., Int. J. Cancer 69, 212-217. 1996; Olivero et al., Br. J. Cancer 74, 1862-1868 1996; Suzuki et al., Hepatology 20, 1231-1236 1994). In addition, overexpression of mRNA has been observed in hepatocellular carcinoma, gastric carcinoma, and colorectal carcinoma (Boix et al., Hepatology 19, 88-91 1994; Kuniyasu et al., Int. J. Cancer 55, 72-75 1993; Liu et al., Oncogene 7, 181-185 1992).

A number of mutations in the kinase domain of Met have been found in renal papillary carcinoma which leads to constitutive receptor activation (Olivero et al., Int. J. Cancer 82, 640-643 1999; Schmidt et al., Nat. Genet. 16, 68-73 1997; Schmidt et al., Oncogene 18, 2343-2350 1999). These activating mutations confer constitutive Met tyrosine phosphorylation and result in MAPK activation, focus formation, and tumorigenesis (Jeffers et al., Proc. Natl. Acad. Sci. USA 94, 11445-11450 1997). In addition, these mutations enhance cell motility and invasion (Giordano et al., 2000; Lorenzato et al., Cancer Res. 62, 7025-7030 2002). HGF-dependent Met activation in transformed cells mediates increased motility, scattering, and migration which eventually leads to invasive tumor growth and metastasis (Jeffers et al., Mol. Cell Biol. 16, 1115-1125 1996; Meiners et al., Oncogene 16, 9-20 1998).

Met is a member of the subfamily of RTKs which include Ron and Sea (Maulik et al., Cytokine Growth Factor Rev. 13, 41-59 2002). Prediction of the extracellular domain structure of Met suggests shared homology with the semaphorins and plexins. The N-terminus of Met contains a Sema domain of approximately 500 amino acids that is conserved in all semaphorins and plexins. The semaphorins and plexins belong to a large family of secreted and membrane-bound proteins first described for their role in neural development (Van Vactor and Lorenz, Curr. Biol. 9, R201-204 1999). However, semaphorin overexpression has been correlated with tumor invasion and metastasis. A cysteine-rich PSI domain (also referred to as a Met Related Sequence domain) found in plexins, semaphorins, and integrins lies adjacent to the Sema domain followed by four IPT repeats that are immunoglobulin-like regions found in plexins and transcription factors. A recent study suggests that the Met Sema domain is sufficient for HGF and heparin binding (Gherardi et al., (2003). Functional map and domain structure of Met, the product of the c-Met protooncogene and receptor for hepatocyte growth factor/scatter factor. Proc. Nall. Acad. Sci. USA 2003). Furthermore, Kong-Beltran et al. (Cancer Cell (2004), 6:61-73) have reported that the Sema domain of Met is necessary for receptor dimerization and activation.

C-Met, a transmembrane receptor tyrosine kinase, plays a key role in malignant transformation of epithelial cells by activating signal transduction pathways essential for cellular proliferation, survival, migration and invasion. C-Met overexpression, with or without gene amplification, has been reported in primary breast cancers and correlate with poor prognosis. C-Met signaling inhibition, such as tyrosine kinase inhibitors (TKIs), usually not sufficient for sustained treatment efficacy. Therefore, we believe that antibody drug conjugates (ADCs) offer the promise and potential of delivering potent anti-tumor activity with the advantage of reduced side effects.

### SUMMARY

The present disclosure provides and antibody drug conjugate (ADC) having an IgG antibody that binds to a c-Met target conjugated at both Cys sites in the hinge region of an IgG antibody. The present disclosure further provides a method for treating a breast cancer comprising providing an effective amount of a c-Met ADC.

We generated antibody drug conjugates containing a novel human anti-c-Met antibody (STI-0602) (described in U.S. patent application Ser. No. 13/924,492 filed 21 Jun. 2013, the disclosure of which is incorporated by reference herein) with either a tubulin inhibitor or DNA damaging agent, such as doxorubicin analogs. The ADC conjugates retained binding affinity and showed potent cell killing in a variety of c-Met positive cell lines.

The present disclosure provides an antibody drug conjugate (ADC) composition comprising an IgG antibody that binds to c-Met, a conjugation linker moiety that binds to both Cys residues in the hinge region of an IgG antibody and a toxin moiety. Preferably, the toxin moiety is a tubulin inhibitor or a doxorubicin analog. Preferably, the antibody is an IgG antibody from the H8 (heavy/light SEQ ID NOs 19/20) family or is a B12 (heavy/light SEQ ID NOs 7/8), wherein the H8 antibody family is selected from the group consisting of H8-A2, H8-9, H8-9EE8L3, H8-C1, H8-D4, H8-D5, H8-D6, H8-D10, H8-E5, H8-G7, H8-H6, H8-2A2, H8-2B1, H8-2B2, H8-2B4, H8-2B7, H8-A7P, H8-9EH11L, H8-9EH11L, and H8-6AG2H3. Preferably, the conjugated toxin is

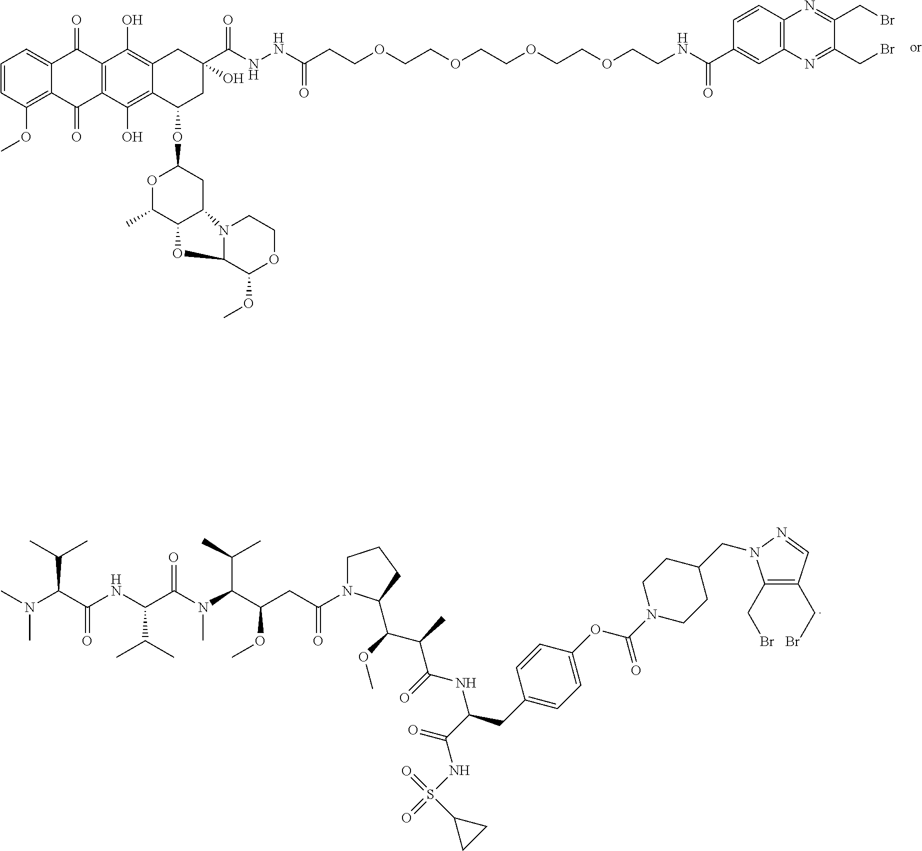

The present disclosure provides a method for treating breast cancer, comprising administering an effective amount of an antibody drug conjugate (ADC) composition comprising an IgG antibody that binds to c-Met, a conjugation linker moiety that binds to both Cys residues in the hinge region of an IgG antibody and a toxin moiety. Preferably, the toxin moiety is a tubulin inhibitor or a doxorubicin analog. Preferably, the antibody is an IgG antibody from the H8 (heavy/light SEQ ID NOs 19/20) family or is a B12 (heavy/light SEQ ID NOs 7/8), wherein the H8 antibody family is selected from the group consisting of H8-A2, H8-9, H8-9EE8L3, H8-C1, H8-D4, H8-D5, H8-D6, H8-D10, H8-E5, H8-G7, H8-H6, H8-2A2, H8-2B1, H8-2B2, H8-2B4, H8-2B7, H8-A7P, H8-9EH11L, H8-9EH11L, and H8-6AG2H3. Preferably, the conjugated toxin is

or

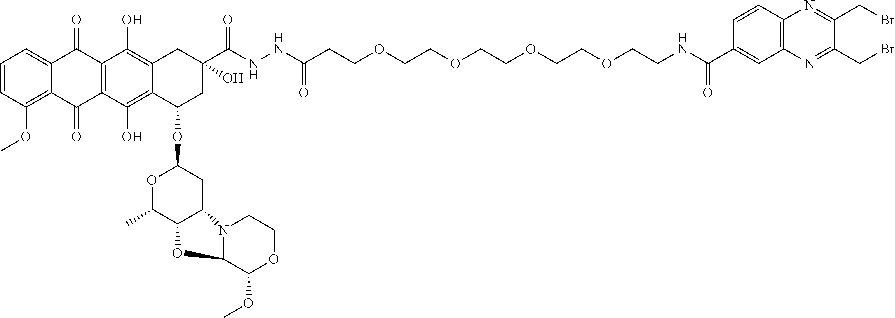 [SMILES: COc1cccc2c1C(=O)c1c(O)c3c(c(O)c1C2=O)C[C@@](O)(C(=O)NNC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1]

0276 Structure for conjugation and drug

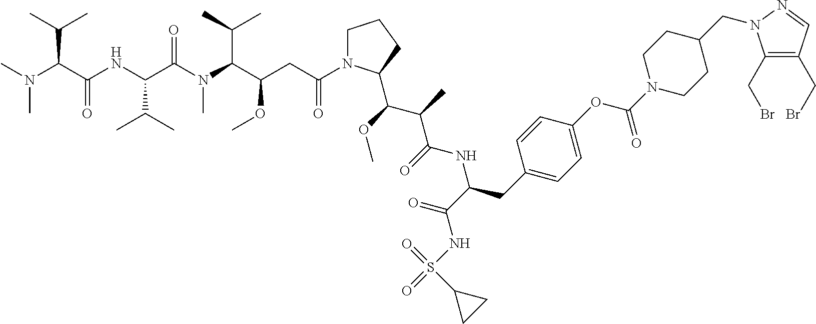

0174 Structure for conjugation and drug.

### BRIEF DESCRIPTION OF THE FIGURES

FIG. 1 shows the effects of treatment with anti-cMet IgG1 antibody STI-0602 described herein with MDA-MB-231 and HS578T cells. The cells were plated at 10K cells/well overnight in complete RPMI and then treated with STI-0602 antibody in serum-free RPMI for 4 hours, starting at an initial concentration of 10 μg/ml, and the serially diluted 1:5 for a total of 8 different concentrations. After 4 hours, cells were stimulated with 50 ng/ml HGF diluted in serum-free RPMI for 15 minutes (media controls were left unstimulated). Cells were then lysed and frozen at −80° C. freezer overnight and phosphorylated c-Met levels were determined using a Cell Signaling ELISA kit.

FIG. 2 shows a comparison of in vivo results achieved comparing 2 c-Met ADC doses (3 mg/kg and 10 mg/kg) to the c-Met antibody alone (STI-0602, also called H8-A2 and disclosed herein as heavy chain SEG ID NO.25 and light chain SEQ ID NO. 26) and control ADC The experimental detail is described in Example 1.

FIG. 3 shows a comparison of in vivo results achieved comparing 2 c-Met ADC doses (3 mg/kg and 10 mg/kg) to the c-Met antibody alone and control ADC. The experimental detail is described in Example 1.

FIG. 4 is a table showing the experimental detail described in Example 1. FIGS. 2 and 3 show the in vivo results.

FIG. 5 shows a graph of three different ADC's, each with the STI 0602 anti c-Met IgG1 antibody and different toxins retain their binding affinities to c-Met TNBC cells. Mice were given a single iv dose at 10 mg/kg. Mean concentrations of total antibody and ADC in serum were determined by ELISA.

FIG. 6 shows a graph of an ADC and a control STI 0602 anti c-Met IgG1 antibody have similar pharmacokinetic properties in mice. Mice were given a single iv dose at 10 mg/kg. Mean concentrations of total antibody and ADC in serum were determined by ELISA.

FIG. 7 shows anti-c-Met ADC's specifically inhibit c-Met TNBC cell proliferation. In the top panel shows inhibition of cell proliferation in HS578T and the lower panel T47D. Cells were plated at 4000 per well and treated with one of the three ADC's. After incubation for 4 days, proliferation was measured using a Cell Titer Glo assay.

FIG. 8 shows that the anti-c-Met IgG1 antibody was internalized in a triple negative breast cancer cell line. Internalization of anti c-Met antibody IgG1 H8-A2 (also called STI-0602 SEQ ID NO. 25/SEQ ID NO. 26) in MDA-MB468. MDA-MB468 were plated overnight and incubated with 1 μg/ml anti-C-Met H8A2 antibody for 180 min at 37° C. Cells were stained with anti-AF488.

FIG. 9 shows an in vivo comparison of two doses of c-Met 0174 ADC at 3 mg/kg and 10 mg/kg and vehicle control with a c-Met antibody.

FIG. 10 shows a single 1 mg/kg much lower dose of c-Met 0276 ADC and vehicle control. Tumor volume was measured twice weekly in all of the mice. FIG. 10 provides these comparative data and it shows a significant effect for the lower 1 mg/kg dose when measuring tumor volume.

FIGS. 11A and 11B show that there was no significant weight change for these mice with either c-Met 0276 ADC (FIG. 11A) or c-Met 0174 ADC (FIG. 11B), compared to vehicle controls. These data suggest that there was no observed toxicity from either ADC tested.

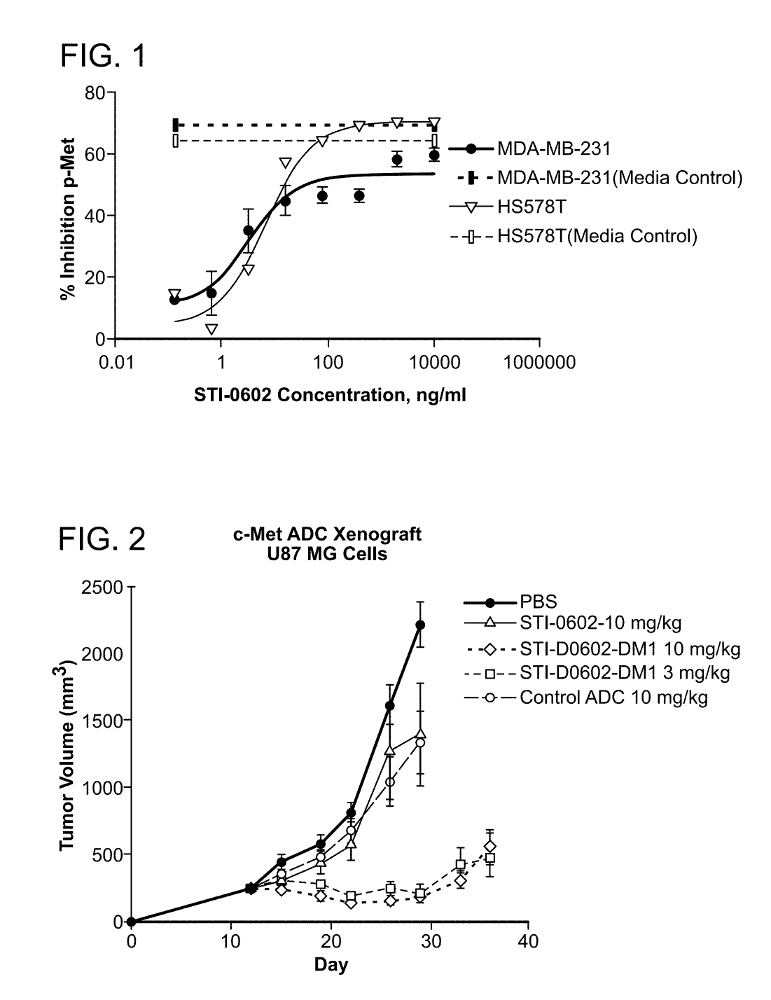

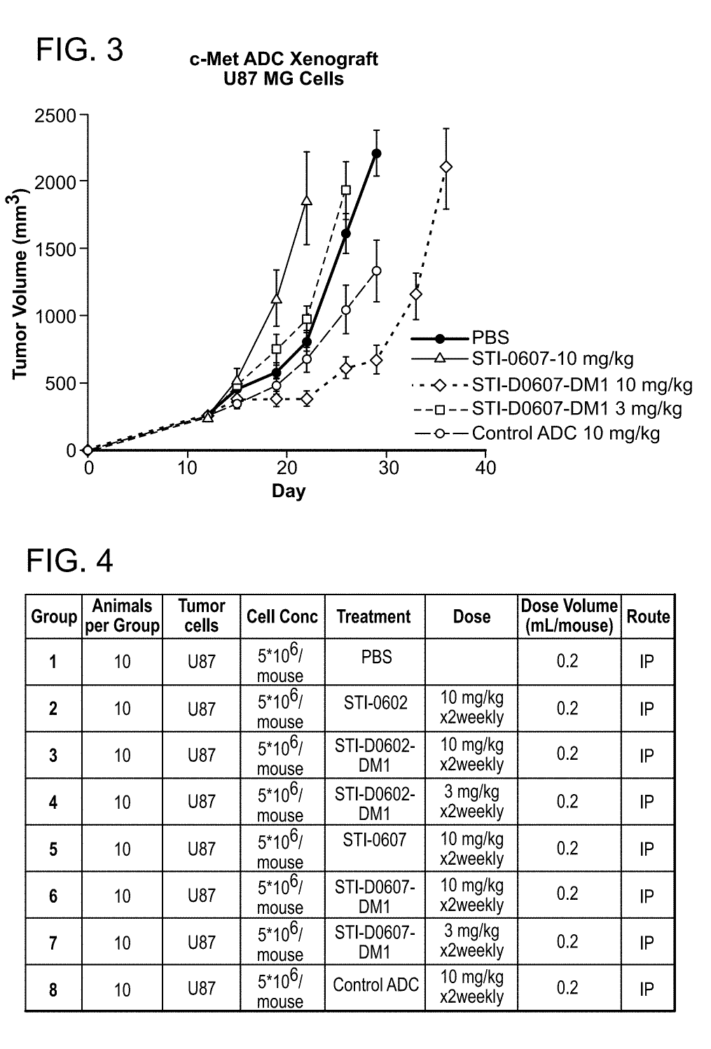

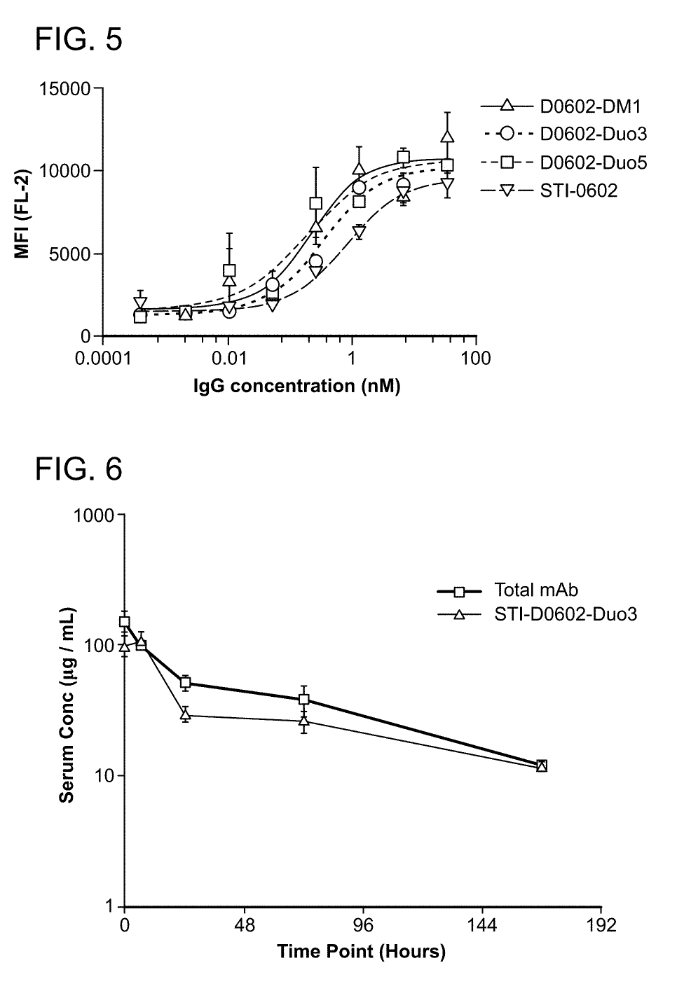

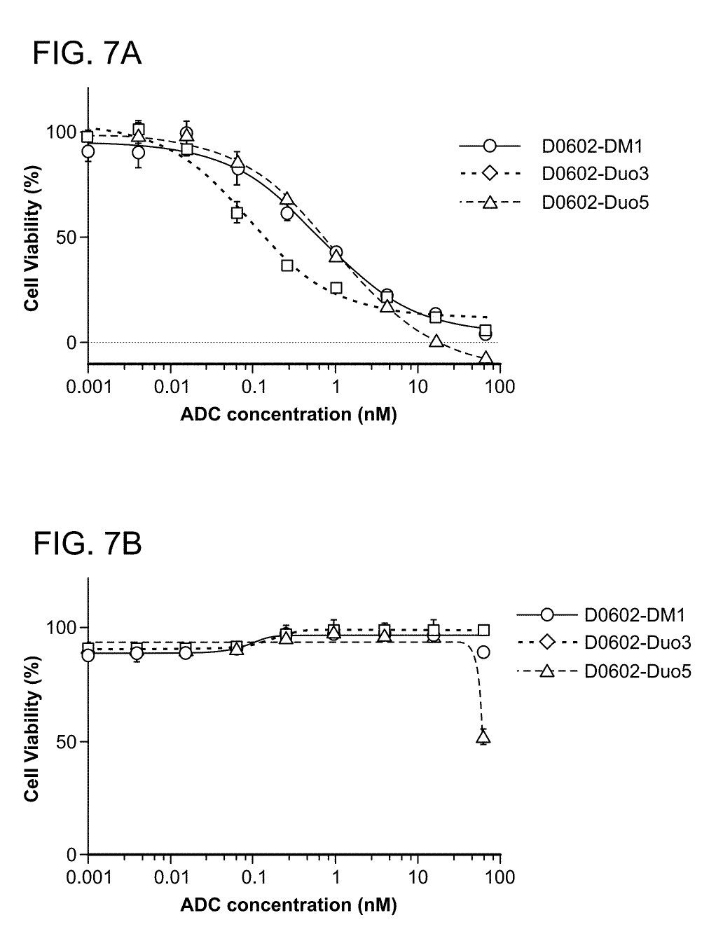

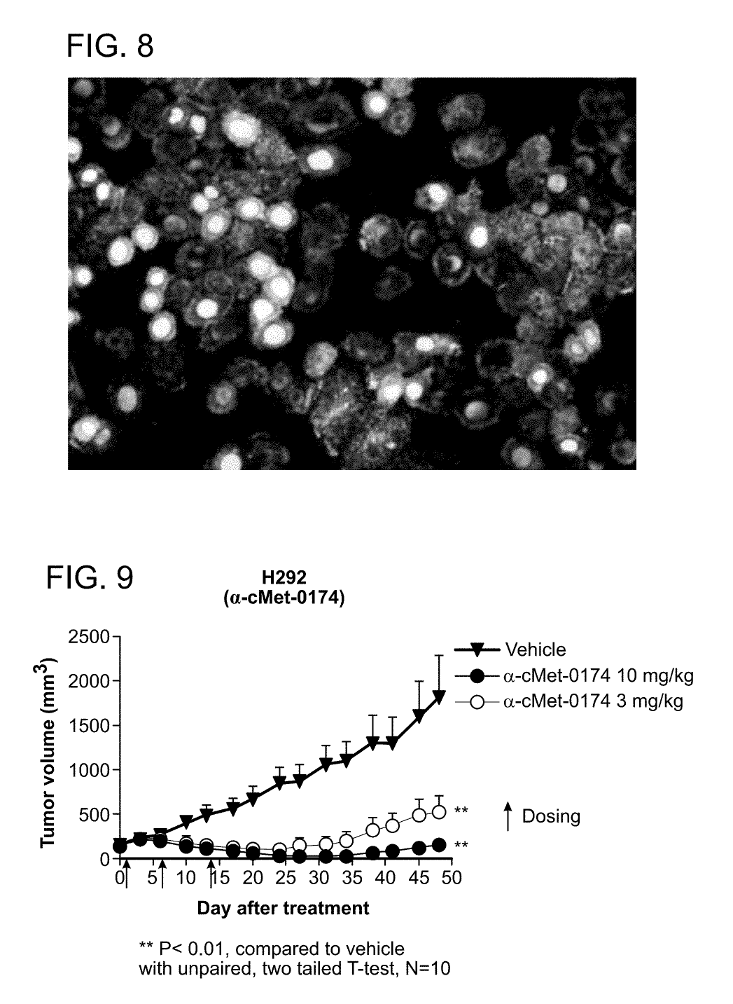

### DETAILED DESCRIPTION

### Antibody Component

The present disclosure provides a fully human antibody of an IgG class that binds to a c-Met epitope with a binding affinity of at least 10⁻⁶M, which has a heavy chain variable domain sequence that is at least 95% identical to the amino acid sequences selected from the group consisting of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 24, SEQ ID NO. 25, SEQ ID NO. 27, SEQ ID NO. 29, SEQ ID NO. 31, SEQ ID NO. 32, SEQ ID NO. 33, SEQ ID NO. 34, SEQ ID NO. 36, SEQ ID NO. 37, SEQ ID NO. 40, SEQ ID NO. 42, SEQ ID NO. 44, SEQ ID NO. 45, SEQ ID NO. 47, SEQ ID NO. 49, SEQ ID NO. 51, SEQ ID NO. 53, SEQ ID NO. 56, SEQ ID NO. 58, SEQ ID NO. 59, SEQ ID NO. 61, SEQ ID NO. 63, SEQ ID NO. 65, SEQ ID NO. 69, SEQ ID NO. 71, SEQ ID NO. 74, SEQ ID NO. 75, SEQ ID NO. 76, SEQ ID NO. 79, SEQ ID NO. 80, SEQ ID NO. 82, SEQ ID NO. 84, SEQ ID NO. 86, SEQ ID NO. 88, SEQ ID NO. 90, SEQ ID NO. 92, and combinations thereof, and that has a light chain variable domain sequence that is at least 95% identical to the amino acid sequences selected from the group consisting of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 23, SEQ ID NO. 26, SEQ ID NO. 28, SEQ ID NO. 30, SEQ ID NO. 35, SEQ ID NO. 38, SEQ ID NO. 39, SEQ ID NO. 41, SEQ ID NO. 43, SEQ ID NO. 46, SEQ ID NO. 48, SEQ ID NO. 50, SEQ ID NO. 52, SEQ ID NO. 54, SEQ ID NO. 55, SEQ ID NO. 57, SEQ ID NO. 60, SEQ ID NO. 62, SEQ ID NO. 64, SEQ ID NO. 66, SEQ ID NO. 67, SEQ ID NO. 68, SEQ ID NO. 70, SEQ ID NO. 72, SEQ ID NO. 73, SEQ ID NO. 77, SEQ ID NO. 78, SEQ ID NO. 81, SEQ ID NO. 83, SEQ ID NO. 85, SEQ ID NO. 87, SEQ ID NO. 89, SEQ ID NO. 91, SEQ ID NO. 93, and combinations thereof. Preferably, the fully human antibody has both a heavy chain and a light chain wherein the antibody has a heavy chain/light chain variable domain sequence selected from the group consisting of SEQ ID NO. 1/SEQ ID NO. 2 (called A1 herein), SEQ ID NO. 3/SEQ ID NO. 4 (called A2 herein), SEQ ID NO. 5/SEQ ID NO. 6 (called A8 herein), SEQ ID NO. 7/SEQ ID NO. 8 (called B12 herein), SEQ ID NO. 9/SEQ ID NO. 10 (called D6 herein), SEQ ID NO. 11/SEQ ID NO. 12 (called E1 herein), SEQ ID NO. 13/SEQ ID NO. 14 (called E6 herein), SEQ ID NO. 15/SEQ ID NO. 16 (called F3 herein), SEQ ID NO. 17/SEQ ID NO. 18 (called H6 herein), SEQ ID NO. 19/SEQ ID NO. 20 (called H8 herein), SEQ ID NO. 21/SEQ ID NO. 22 (called H8-9 herein), SEQ ID NO. 21/SEQ ID NO. 23 (called H8-9EE8L3 herein), SEQ ID NO. 24/SEQ ID NO. 22 (called H8-G3S herein), SEQ ID NO. 25/SEQ ID NO. 26 (called H8-A2 herein), SEQ ID NO. 27/SEQ ID NO. 28 (called H8-B6 herein), SEQ ID NO. 29/SEQ ID NO. 23 (called H8-C1 herein), SEQ ID NO. 24/SEQ ID NO. 30 (called H8-D4 herein), SEQ ID NO. 31/SEQ ID NO. 23 (called H8-D5 herein), SEQ ID NO. 24/SEQ ID NO. 23 (called H8-D6 herein), SEQ ID NO. 32/SEQ ID NO. 23 (called H8-D10 herein), SEQ ID NO. 33/SEQ ID NO. 22 (called H8-E5 herein), SEQ ID NO. 34/SEQ ID NO. 22 (called H8-G7 herein), SEQ ID NO. 24/SEQ ID NO. 35 (called H8-G9 herein), SEQ ID NO. 36/SEQ ID NO. 26 (called H8-H6 herein), SEQ ID NO. 29/SEQ ID NO. 22 (called H8-2A2 herein), SEQ ID NO. 37/SEQ ID NO. 38 (called H8-2B1 herein), SEQ ID NO. 34/SEQ ID NO. 23 (called H8-2B2 herein), SEQ ID NO. 37/SEQ ID NO. 23 (called H8-2B4 herein), SEQ ID NO. 32/SEQ ID NO. 39 (called H8-2B7 herein), SEQ ID NO. 32/SEQ ID NO. 22 (called H8-A7P herein), SEQ ID NO. 40/SEQ ID NO. 41 (called GCE-A10 herein), SEQ ID NO. 42/SEQ ID NO. 43 (called GCE-A11 herein), SEQ ID NO. 44/SEQ ID NO. 41 (called GCE-A13 herein), SEQ ID NO. 45/SEQ ID NO. 46 (called GCE-A14 herein), SEQ ID NO. 47/SEQ ID NO. 48 (called GCE-A16 herein), SEQ ID NO. 49/SEQ ID NO. 50 (called GCE-A18 herein), SEQ ID NO. 51/SEQ ID NO. 52 (called GCE-B2 herein), SEQ ID NO. 53/SEQ ID NO. 54 (called GCE-B9 herein), SEQ ID NO. 45/SEQ ID NO. 55 (called GCE-B11 herein), SEQ ID NO. 56/SEQ ID NO. 57 (called GCE-B13 herein), SEQ ID NO. 58/SEQ ID NO. 57 (called GCE-B19 herein), SEQ ID NO. 59/SEQ ID NO. 60 (called GCE-BR1 herein), SEQ ID NO. 61/SEQ ID NO. 62 (called GCE-B20 herein), SEQ ID NO. 63/SEQ ID NO. 64 (called GCE-A19 herein), SEQ ID NO. 65/SEQ ID NO. 66 (called GCE-B10 herein), SEQ ID NO. 58/SEQ ID NO. 67 (called GCE-B5 herein), SEQ ID NO. 61/SEQ ID NO. 68 (called GCE-B4 herein), SEQ ID NO. 69/SEQ ID NO. 70 (called GCE-A26 herein), SEQ ID NO. 71/SEQ ID NO. 72 (called GCE-L1A-9 herein), SEQ ID NO. 49/SEQ ID NO. 73 (called GCE-H34-36 herein), SEQ ID NO. 74/SEQ ID NO. 73 (called GCE-H13-1 herein), SEQ ID NO. 61/SEQ ID NO. 73 (called GCE-H13-2 herein), SEQ ID NO. 44/SEQ ID NO. 73 (called GCE-H13-3 herein), SEQ ID NO. 40/SEQ ID NO. 73 (called GCE-H13-4 herein), SEQ ID NO. 75/SEQ ID NO. 73 (called GCE-H13-5 herein), SEQ ID NO. 69/SEQ ID NO. 73 (called GCE-H13-6 herein), SEQ ID NO. 76/SEQ ID NO. 73 (called GCE-H13-8 herein), SEQ ID NO. 21/SEQ ID NO. 77 (called H8-9EH11L herein), SEQ ID NO. 21/SEQ ID NO. 78 (called H8-9EG11L herein), SEQ ID NO. 79/SEQ ID NO. 20 (called H8-6AG2H3 herein), SEQ ID NO. 80/SEQ ID NO. 81 (called A1-2 herein), SEQ ID NO. 82/SEQ ID NO. 83 (called A1-4 herein), SEQ ID NO. 84/SEQ ID NO. 85 (called A1-6 herein), SEQ ID NO. 86/SEQ ID NO. 87 (called A1-8 herein), SEQ ID NO. 88/SEQ ID NO. 89 (called A1-9 herein), SEQ ID NO. 90/SEQ ID NO. 91 (called A1-24 herein), SEQ ID NO. 92/SEQ ID NO. 93 (called A1-32 herein), and combinations thereof.

The present disclosure provides a fully human antibody Fab fragment, having a variable domain region from a heavy chain and a variable domain region from a light chain, wherein the heavy chain variable domain sequence that is at least 95% identical to the amino acid sequences selected from the group consisting of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 24, SEQ ID NO. 25, SEQ ID NO. 27, SEQ ID NO. 29, SEQ ID NO. 31, SEQ ID NO. 32, SEQ ID NO. 33, SEQ ID NO. 34, SEQ ID NO. 36, SEQ ID NO. 37, SEQ ID NO. 40, SEQ ID NO. 42, SEQ ID NO. 44, SEQ ID NO. 45, SEQ ID NO. 47, SEQ ID NO. 49, SEQ ID NO. 51, SEQ ID NO. 53, SEQ ID NO. 56, SEQ ID NO. 58, SEQ ID NO. 59, SEQ ID NO. 61, SEQ ID NO. 63, SEQ ID NO. 65, SEQ ID NO. 69, SEQ ID NO. 71, SEQ ID NO. 74, SEQ ID NO. 75, SEQ ID NO. 76, SEQ ID NO. 79, SEQ ID NO. 80, SEQ ID NO. 82, SEQ ID NO. 84, SEQ ID NO. 86, SEQ ID NO. 88, SEQ ID NO. 90, SEQ ID NO. 92, and combinations thereof, and that has a light chain variable domain sequence that is at least 95% identical to the amino acid sequences selected from the group consisting of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 23, SEQ ID NO. 26, SEQ ID NO. 28, SEQ ID NO. 30, SEQ ID NO. 35, SEQ ID NO. 38, SEQ ID NO. 39, SEQ ID NO. 41, SEQ ID NO. 43, SEQ ID NO. 46, SEQ ID NO. 48, SEQ ID NO. 50, SEQ ID NO. 52, SEQ ID NO. 54, SEQ ID NO. 55, SEQ ID NO. 57, SEQ ID NO. 60, SEQ ID NO. 62, SEQ ID NO. 64, SEQ ID NO. 66, SEQ ID NO. 67, SEQ ID NO. 68, SEQ ID NO. 70, SEQ ID NO. 72, SEQ ID NO. 73, SEQ ID NO. 77, SEQ ID NO. 78, SEQ ID NO. 81, SEQ ID NO. 83, SEQ ID NO. 85, SEQ ID NO. 87, SEQ ID NO. 89, SEQ ID NO. 91, SEQ ID NO. 93, and combinations thereof. Preferably, the fully human antibody Fab fragment has both a heavy chain variable domain region and a light chain variable domain region wherein the antibody has a heavy chain/light chain variable domain sequence selected from the group consisting of SEQ ID NO. 1/SEQ ID NO. 2, SEQ ID NO. 3/SEQ ID NO. 4, SEQ ID NO. 5/SEQ ID NO. 6, SEQ ID NO. 7/SEQ ID NO. 8, SEQ ID NO. 9/SEQ ID NO. 10, SEQ ID NO. 11/SEQ ID NO. 12, SEQ ID NO. 13/SEQ ID NO. 14, SEQ ID NO. 15/SEQ ID NO. 16, SEQ ID NO. 17/SEQ ID NO. 18, SEQ ID NO. 19/SEQ ID NO. 20, SEQ ID NO. 21/SEQ ID NO. 22, SEQ ID NO. 21/SEQ ID NO. 23, SEQ ID NO. 24/SEQ ID NO. 22, SEQ ID NO. 25/SEQ ID NO. 26, SEQ ID NO. 27/SEQ ID NO. 28, SEQ ID NO. 29/SEQ ID NO. 23, SEQ ID NO. 24/SEQ ID NO. 30, SEQ ID NO. 31/SEQ ID NO. 23, SEQ ID NO. 24/SEQ ID NO. 23, SEQ ID NO. 32/SEQ ID NO. 23, SEQ ID NO. 33/SEQ ID NO. 22, SEQ ID NO. 34/SEQ ID NO. 22, SEQ ID NO. 24/SEQ ID NO. 35, SEQ ID NO. 36/SEQ ID NO. 26, SEQ ID NO. 29/SEQ ID NO. 22, SEQ ID NO. 37/SEQ ID NO. 38, SEQ ID NO. 34/SEQ ID NO. 23, SEQ ID NO. 37/SEQ ID NO. 23, SEQ ID NO. 32/SEQ ID NO. 39, SEQ ID NO. 32/SEQ ID NO. 22, SEQ ID NO. 40/SEQ ID NO. 41, SEQ ID NO. 42/SEQ ID NO. 43, SEQ ID NO. 44/SEQ ID NO. 41, SEQ ID NO. 45/SEQ ID NO. 46, SEQ ID NO. 47/SEQ ID NO. 48, SEQ ID NO. 49/SEQ ID NO. 50, SEQ ID NO. 51/SEQ ID NO. 52, SEQ ID NO. 53/SEQ ID NO. 54, SEQ ID NO. 45/SEQ ID NO. 55, SEQ ID NO. 56/SEQ ID NO. 57, SEQ ID NO. 58/SEQ ID NO. 57, SEQ ID NO. 59/SEQ ID NO. 60, SEQ ID NO. 61/SEQ ID NO. 62, SEQ ID NO. 63/SEQ ID NO. 64, SEQ ID NO. 65/SEQ ID NO. 66, SEQ ID NO. 58/SEQ ID NO. 67, SEQ ID NO. 61/SEQ ID NO. 68, SEQ ID NO. 69/SEQ ID NO. 70, SEQ ID NO. 71/SEQ ID NO. 72, SEQ ID NO. 49/SEQ ID NO. 73, SEQ ID NO. 74/SEQ ID NO. 73, SEQ ID NO. 61/SEQ ID NO. 73, SEQ ID NO. 44/SEQ ID NO. 73, SEQ ID NO. 40/SEQ ID NO. 73, SEQ ID NO. 75/SEQ ID NO. 73, SEQ ID NO. 69/SEQ ID NO. 73, SEQ ID NO. 76/SEQ ID NO. 73, SEQ ID NO. 21/SEQ ID NO. 77, SEQ ID NO. 21/SEQ ID NO. 78, SEQ ID NO. 79/SEQ ID NO. 20, SEQ ID NO. 80/SEQ ID NO. 81, SEQ ID NO. 82/SEQ ID NO. 83, SEQ ID NO. 84/SEQ ID NO. 85, SEQ ID NO. 86/SEQ ID NO. 87, SEQ ID NO. 88/SEQ ID NO. 89, SEQ ID NO. 90/SEQ ID NO. 91, SEQ ID NO. 92/SEQ ID NO. 93, and combinations thereof.

The present disclosure provides a single chain human antibody, having a variable domain region from a heavy chain and a variable domain region from a light chain and a peptide linker connection the heavy chain and light chain variable domain regions, wherein the heavy chain variable domain sequence that is at least 95% identical to the amino acid sequences selected from the group consisting of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 24, SEQ ID NO. 25, SEQ ID NO. 27, SEQ ID NO. 29, SEQ ID NO. 31, SEQ ID NO. 32, SEQ ID NO. 33, SEQ ID NO. 34, SEQ ID NO. 36, SEQ ID NO. 37, SEQ ID NO. 40, SEQ ID NO. 42, SEQ ID NO. 44, SEQ ID NO. 45, SEQ ID NO. 47, SEQ ID NO. 49, SEQ ID NO. 51, SEQ ID NO. 53, SEQ ID NO. 56, SEQ ID NO. 58, SEQ ID NO. 59, SEQ ID NO. 61, SEQ ID NO. 63, SEQ ID NO. 65, SEQ ID NO. 69, SEQ ID NO. 71, SEQ ID NO. 74, SEQ ID NO. 75, SEQ ID NO. 76, SEQ ID NO. 79, SEQ ID NO. 80, SEQ ID NO. 82, SEQ ID NO. 84, SEQ ID NO. 86, SEQ ID NO. 88, SEQ ID NO. 90, SEQ ID NO. 92, and combinations thereof, and that has a light chain variable domain sequence that is at least 95% identical to the amino acid sequences selected from the group consisting of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 23, SEQ ID NO. 26, SEQ ID NO. 28, SEQ ID NO. 30, SEQ ID NO. 35, SEQ ID NO. 38, SEQ ID NO. 39, SEQ ID NO. 41, SEQ ID NO. 43, SEQ ID NO. 46, SEQ ID NO. 48, SEQ ID NO. 50, SEQ ID NO. 52, SEQ ID NO. 54, SEQ ID NO. 55, SEQ ID NO. 57, SEQ ID NO. 60, SEQ ID NO. 62, SEQ ID NO. 64, SEQ ID NO. 66, SEQ ID NO. 67, SEQ ID NO. 68, SEQ ID NO. 70, SEQ ID NO. 72, SEQ ID NO. 73, SEQ ID NO. 77, SEQ ID NO. 78, SEQ ID NO. 81, SEQ ID NO. 83, SEQ ID NO. 85, SEQ ID NO. 87, SEQ ID NO. 89, SEQ ID NO. 91, SEQ ID NO. 93, and combinations thereof. Preferably, the fully human single chain antibody has both a heavy chain variable domain region and a light chain variable domain region, wherein the single chain fully human antibody has a heavy chain/light chain variable domain sequence selected from the group consisting of SEQ ID NO. 1/SEQ ID NO. 2, SEQ ID NO. 3/SEQ ID NO. 4, SEQ ID NO. 5/SEQ ID NO. 6, SEQ ID NO. 7/SEQ ID NO. 8, SEQ ID NO. 9/SEQ ID NO. 10, SEQ ID NO. 11/SEQ ID NO. 12, SEQ ID NO. 13/SEQ ID NO. 14, SEQ ID NO. 15/SEQ ID NO. 16, SEQ ID NO. 17/SEQ ID NO. 18, SEQ ID NO. 19/SEQ ID NO. 20, SEQ ID NO. 21/SEQ ID NO. 22, SEQ ID NO. 21/SEQ ID NO. 23, SEQ ID NO. 24/SEQ ID NO. 22, SEQ ID NO. 25/SEQ ID NO. 26, SEQ ID NO. 27/SEQ ID NO. 28, SEQ ID NO. 29/SEQ ID NO. 23, SEQ ID NO. 24/SEQ ID NO. 30, SEQ ID NO. 31/SEQ ID NO. 23, SEQ ID NO. 24/SEQ ID NO. 23, SEQ ID NO. 32/SEQ ID NO. 23, SEQ ID NO. 33/SEQ ID NO. 22, SEQ ID NO. 34/SEQ ID NO. 22, SEQ ID NO. 24/SEQ ID NO. 35, SEQ ID NO. 36/SEQ ID NO. 26, SEQ ID NO. 29/SEQ ID NO. 22, SEQ ID NO. 37/SEQ ID NO. 38, SEQ ID NO. 34/SEQ ID NO. 23, SEQ ID NO. 37/SEQ ID NO. 23, SEQ ID NO. 32/SEQ ID NO. 39, SEQ ID NO. 32/SEQ ID NO. 22, SEQ ID NO. 40/SEQ ID NO. 41, SEQ ID NO. 42/SEQ ID NO. 43, SEQ ID NO. 44/SEQ ID NO. 41, SEQ ID NO. 45/SEQ ID NO. 46, SEQ ID NO. 47/SEQ ID NO. 48, SEQ ID NO. 49/SEQ ID NO. 50, SEQ ID NO. 51/SEQ ID NO. 52, SEQ ID NO. 53/SEQ ID NO. 54, SEQ ID NO. 45/SEQ ID NO. 55, SEQ ID NO. 56/SEQ ID NO. 57, SEQ ID NO. 58/SEQ ID NO. 57, SEQ ID NO. 59/SEQ ID NO. 60, SEQ ID NO. 61/SEQ ID NO. 62, SEQ ID NO. 63/SEQ ID NO. 64, SEQ ID NO. 65/SEQ ID NO. 66, SEQ ID NO. 58/SEQ ID NO. 67, SEQ ID NO. 61/SEQ ID NO. 68, SEQ ID NO. 69/SEQ ID NO. 70, SEQ ID NO. 71/SEQ ID NO. 72, SEQ ID NO. 49/SEQ ID NO. 73, SEQ ID NO. 74/SEQ ID NO. 73, SEQ ID NO. 61/SEQ ID NO. 73, SEQ ID NO. 44/SEQ ID NO. 73, SEQ ID NO. 40/SEQ ID NO. 73, SEQ ID NO. 75/SEQ ID NO. 73, SEQ ID NO. 69/SEQ ID NO. 73, SEQ ID NO. 76/SEQ ID NO. 73, SEQ ID NO. 21/SEQ ID NO. 77, SEQ ID NO. 21/SEQ ID NO. 78, SEQ ID NO. 79/SEQ ID NO. 20, SEQ ID NO. 80/SEQ ID NO. 81, SEQ ID NO. 82/SEQ ID NO. 83, SEQ ID NO. 84/SEQ ID NO. 85, SEQ ID NO. 86/SEQ ID NO. 87, SEQ ID NO. 88/SEQ ID NO. 89, SEQ ID NO. 90/SEQ ID NO. 91, SEQ ID NO. 92/SEQ ID NO. 93, and combinations thereof.

Preparation of cMet-DM1 ADC

Anti-c-Met antibody was buffer exchanged to phosphate buffer, pH from 6.5 to 7.5. Toxin-linker, SMCC-DM1 was dissolved in DMA (Dimethylacetamide) solution and added to antibody solution with Toxin/Antibody ratio from 7 to 10. The antibody-toxin solution was incubated at room temperature overnight. The unconjugated antibody was removed either gel-filtration chromatography or centrifugation filtration. The cMet-DM1 was characterized by HPLC. The drug antibody ratio (DAR) was calculated based on UV-VIS of cMet-DM1.

Preparation of cMet-Duo3
Anti-cMet antibody was reduced by TCEP (tris(2-carboxyethyl)phosphine), up to 20 mM. The excess of TCEP was removed by gel-filtration chromatography or centrifugal filtration. Toxin-Duo3-linker was dissolved in DMA solution and added to the reduced antibody with Toxin/antibody ratio from 4.5 to 6. After few hours' incubation at room temperature, the unconjugated Duo3-linker was removed by gel-filtration chromatography or centrifugal filtration. The cMet-Duo3 was characterized by HPLC. The drug antibody ratio (DAR) was calculated based on UV-VIS or HIC-HPLC.

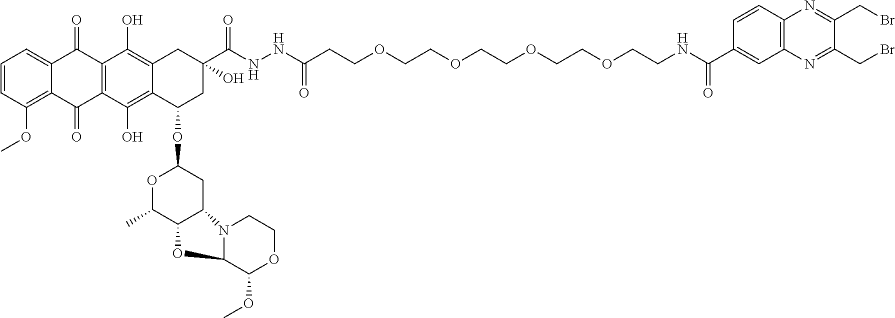 [SMILES: COc1cccc2c1C(=O)c1c(O)c3c(c(O)c1C2=O)C[C@@](O)(C(=O)NNC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1]

Structure of compound 030-0260 and preparation as follows:

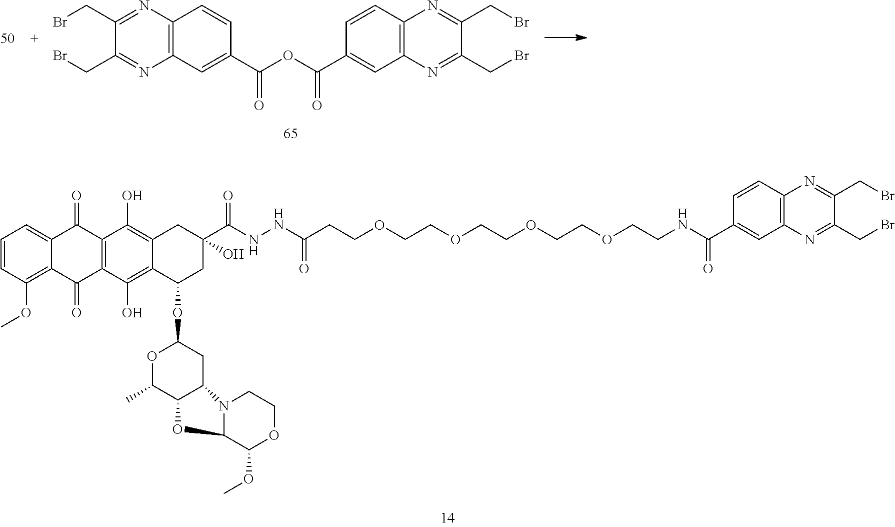 [SMILES: O=C(OC(=O)c1ccc2nc(CBr)c(CBr)nc2c1)c1ccc2nc(CBr)c(CBr)nc2c1 ; COc1cccc2c1C(=O)c1c(O)c3c(c(O)c1C2=O)C[C@@](O)(C(=O)NNC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1]

To a solution of compound 50 (18 mg, 0.02 mmol) in DCM (2 mL) was added compound 65 (15 mg), followed by DIEA (5 μL). The mixture was stirred at room temperature for 10 min. The reaction was then diluted with DCM (30 mL) and washed with aq. saturated NaHCO₃. The organic layer was concentrated and residue was purified by RP-HPLC to give compound 14 as a red solid after lyophilization (7 mg, 29%). MS m/z 1231.3 (M+H).

Structure of compound 030-0174

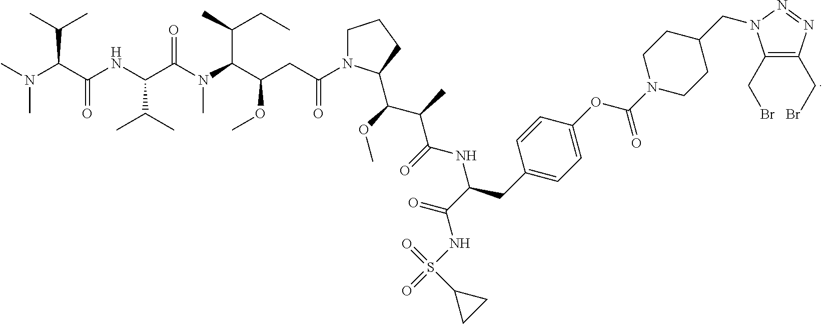 [SMILES: CC[C@H](C)[C@@H]([C@@H](CC(=O)N1CCC[C@H]1[C@H](OC)[C@@H](C)C(=O)N[C@@H](Cc1ccc(OC(=O)N2CCC(Cn3nnc(CBr)c3CBr)CC2)cc1)C(=O)NS(=O)(=O)C1CC1)OC)N(C)C(=O)[C@@H](NC(=O)[C@H](C(C)C)N(C)C)C(C)C]

The synthesis of this compound was described as compound 8.
Preparation of compound 8

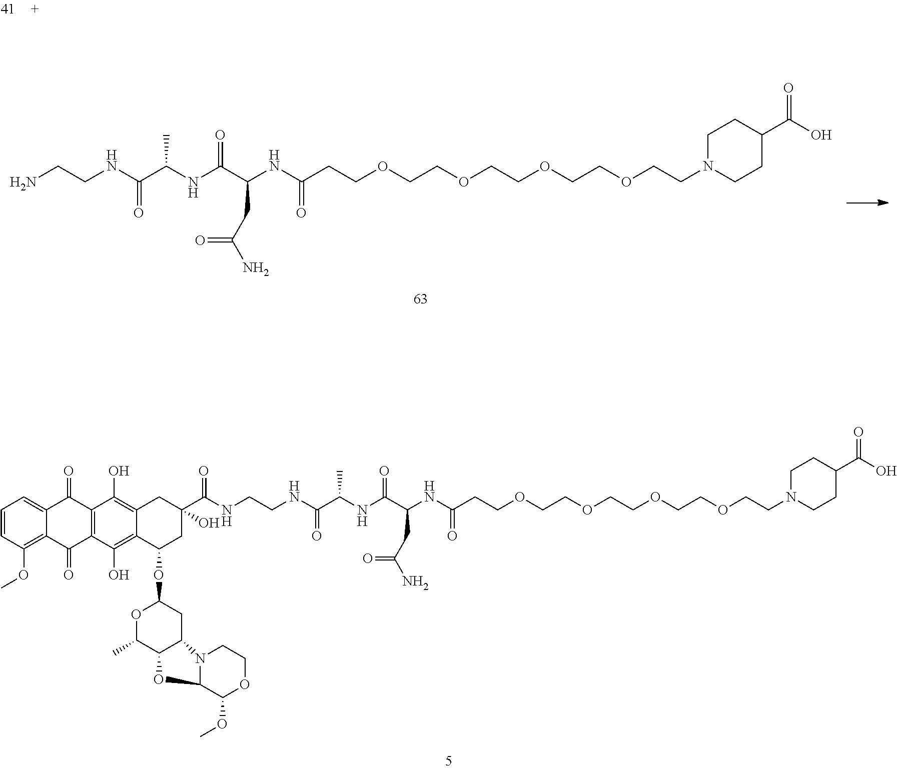 [SMILES: C[C@H](NC(=O)[C@H](CC(N)=O)NC(=O)CCOCCOCCOCCOCCN1CCC(C(=O)O)CC1)C(=O)NCCN ; COc1cccc2c1C(=O)c1c(O)c3c(c(O)c1C2=O)C[C@@](O)(C(=O)NCCNC(=O)[C@H](C)NC(=O)[C@H](CC(N)=O)NC(=O)CCOCCOCCOCCOCCN1CCC(C(=O)O)CC1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (75 μL), and amine TFA 63 (86 mg, 0.12 mmol). The mixture was stirred at room temperature for 3 h, then diluted with DCM (40 mL). The mixture was washed with brine. The organic layer was dried and evaporated to dryness. The residue was purified by column (silica gel, DCM:MeOH, 9:1) to give compound 8 (63 mg, 52%). MS m/z 1214.5 (M+H).

### Example 1

Upon receipt, animals were housed 5 mice per cage in a room with a controlled environment. Animals were provided rodent chow and water ad libitum. Acclimation of the mice to laboratory conditions was at least 72 hours prior to the start of cell administration and dosing. During the acclimation period, the animals' health status was determined. Only animals that are observed to be healthy prior to study initiation were used.

This example provides an in vivo experiment comparing treatment of mice with control (PBS), anti-c-Met IgG1 antibody (STI-0602 and STI-0607) and an ADC variant of both antibodies. The procedure first does a tumor cell inoculation & establishment of tumors:

a. U87 cells were cultured with 10% FBS U87 medium (EMEM) and harvested with 0.05% trypsin. Cells were washed 2 times with serum-free EMEM, counted, and resuspended at 5×10⁶ cells in 0.2 mL or, 25×10⁶ cells/mL in a 1:1 mix of serum-free EMEM and matrigel and injected subcutaneously into the upper right flank of each mouse.
b. Tumor growth was monitored by tumor volume measurement using a digital caliper starting Day 6-9 after inoculation, 2 times per week thereafter and prior to study termination.
c. Tumors were measured with digital calipers. The length (the longest dimension) and the width (the distance perpendicular to and in the same plane as the length) were measured. The formula for calculating tumor volume was TV (mm3)=½×L×W2.

### Treatments:

a. Once tumors were staged to the desired volume (average from 200 to 300 mm3), animals were randomized and mice with very large or small tumors culled. Mice were divided into 8 groups of 10 mice each, randomized by tumor volume.
b. Mice were treated with either vehicle or Test Article according to FIG. 4. Mice received a total of 5 doses.
c. Tumor responses were monitored and study terminated once clear treatment trends are established and/or when tumor load in vehicle-treated mice reaches IACUC protocol limits (2000 mm³).

### Example 2

This example is an in vivo experiment comparing two disclosed c-Met ADCs in vivo with mice having (H292 non-small cell lung cancer line). ADC or vehicle control was administered iv to the tail in three weekly doses. FIG. 9 shows two different doses of c-Met 0174 ADC at 3 mg/kg and 10 mg/kg and vehicle control. Tumor volume was measured twice weekly in all of the mice.

FIG. 9 provides these comparative data and it shows a significant dose-dependent effect when measuring tumor volume. FIG. 11B shows that there was no significant weight change for these mice, compared to vehicle control. These data suggest that there was no observed toxicity from the ADC.

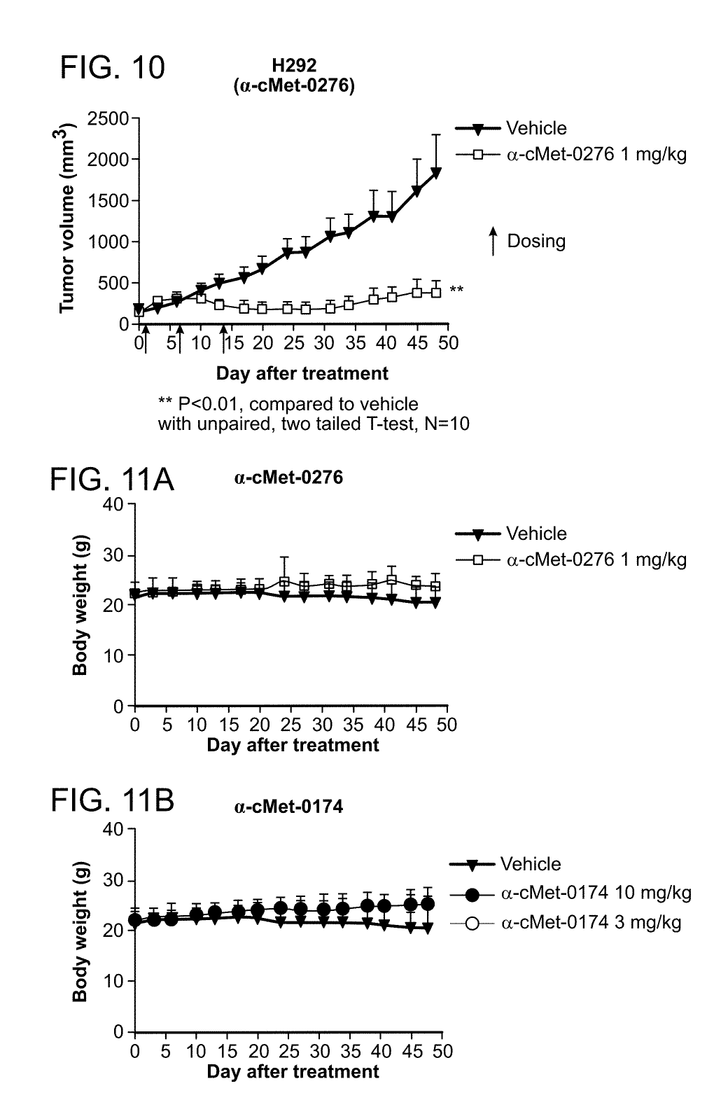

FIG. 10 shows a single 1 mg/kg much lower dose of c-Met 0276 ADC at and vehicle control. Tumor volume was measured twice weekly in all of the mice. FIG. 10 provides these comparative data and it shows a significant effect for the lower 1 mg/kg dose when measuring tumor volume. FIG. 11A shows that there was no significant weight change for these mice, compared to vehicle control. These data suggest that there was no observed toxicity from this ADC.

Sequence Listing

| | Heavy chain variable domain region | Light chain variable domain region |
|---|---|---|
| A1 | QVQLQESGPGLVKPSQTLSLTCTVSGGSISSG | LPVLTQPASVSGSPGQSITISCTGTSSD |
| | GYYWSWIRQHPGKGLEWIGEINHSGSTNYNP | VGGYNYVSWYQQHPGKAPKLMIYDVS |
| | SLKSRVTISVDTSKNQFSLKLSSVTAADTAVYY | DRPSGVSTRFSGSKSGNTASLTISGLQ |
| | CARGRDGYDFDPWGQGTLVTVSS SEQ ID | AEDEADYYCSSYRSSSALVVFGGGTK |
| | NO. 1 | LTVL SEQ ID NO. 2 |
| A2 | QVQLQESGPGLVKPSGTLSLTCAVSGGSISRS | LPVLTQPASVSGSPGQSITISCTGTSSD |
| | NWWSWVRQPPGKGLEWIGEVYHSGSTNYNP | VGGYKYVSWYQQHPGKAPKLLIYDVT |
| | SLKSRVTISVDKSKNQFSLKVNSVTAADTAVY | DRPSGVSNRFSGSQSGNTASLTISGLQ |
| | YCARDSDGGYYFDYWGQGTLVTVSS SEQ | TEDEADYYCSSYTDNGALVVFGGGTK |
| | ID NO. 3 | LTVL SEQ ID NO. 4 |
| A8 | QITLKESGAEVKKPGSSVKVSCKASGGTFSSY | SYELMQPASVSGSPGQSITISCTGTSS |
| | GISWVRQAPGQGLEWMGGIIPMFGTANYAQK | DVGGYDHVSWYQQHPGKAPKLMIYAV |
| | FQGRVTITADESTSTAYMELSSLRSEDTAVYY | RNRPSGVPDRFSGSKSGNTASLTISGL |
| | CARDEVAPDYYGSGPSYGMDVWGQGTMVT | QAEDEADYYCSSYTSSLTYVFGTGTKV |
| | VSS SEQ ID NO. 5 | TVL SEQ ID NO. 6 |
| B12 | QVQLVESGAEVKKPGASVKVSCKASGYTFTG | QAVLTQPPSVSGSPGQSITISCTGTSS |
| | YYMHWVRQAPGQGLEWMGWINPNSGNTGY | DVGTFNLVSWYQQHPGKAPKLIIYEVS |
| | AQKFQGRVTMTRNTSISTAYMELSSLRSEDTA | KRPSDVSPRYSGSKSGTTASLTISVLQ |
| | VYYCARRGTTVSFDYWGQGTTVTVSS SEQ | TEDEADYYCCSYTTSSSYVFGIGTKVT |
| | ID NO. 7 | VL SEQ ID NO. 8 |
| D6 | QVQLQQWGAGLLKPSETLSLTCAVYGGSFSG | QSVLTQPPSASGSPGQSVTISCTGTSS |
| | YYWSWIRQPPGKGLEWIGEINHSGSTNYNPS | DVGGYNYVSWYQQHPGKAPKLMIYEV |
| | LKSRVTISVDTSKNQFSLKLSSVTAADTAVYYC | SKRPSGVPDRFSGSKSGNTASLTVSG |
| | ARGRDGYDFDPWGQGTLVTVSS SEQ ID | LQAEDEADYYCSSYAGSNNLVVFGGG |
| | NO. 9 | TQLTVL SEQ ID NO. 10 |
| E1 | QVQLVQSGAEVKKPGASVKVSCKTSGYTFSG | QSVVTQPPSVSGAPGQRVTISCTGSS |
| | DYMHWVRQAPGQGLEWMGWINPNSGGTNY | SNIGAGYDVHWYQQLPGTVPKLLIYGN |
| | AQKFQGRVTMTRDTSISTAYMELSRLRSDDT | SNRPSGVPDRFSGSKSGTSASLAITGL |
| | AVYYCAREPGRDYYYYDGMDVWGQGTTVTV | QAEDEADYYCQSYDSSLSAYVFGTGT |
| | SS SEQ ID NO. 11 | KVTVL SEQ ID NO. 12 |
| E6 | QVQLQQWGAGLLKPSETLSLTCAVYGGSFSG | QAVLTQPASVSGSPGQSITISCTGTRS |
| | YYWSWIRQPPGKGLEWIGEINHSGSTNYNPS | DVGGYNYVSWYQQHPGKAPKLLVYDV |
| | LKSRVTISVDTSKNQFSLKLSSVTAADTAVYYC | SNRPSGVSNRFSGSQSGNTASLTISGL |
| | ARGGRVYSNYYMDVWGKGTTVTVSS SEQ | QTEDEADYYCSSYTDNSALVVFGGGT |
| | ID NO. 13 | KVTVL SEQ ID NO. 14 |
| F3 | QVQLVESGPGLVKPSGTLSLTCAVSGGSISSS | QSVLTQPASVSGSPGQSITISCTGTSS |
| | NWWSWVRQPPGKGLEWIGEIYHSGSTNYNP | DVGGYNYVSWYQQHPGKAPKLLIYDV |
| | SLKSRVTISVDKSKNQFSLKLSSVTAADTAVYY | DSRPSGVSNRFSGSKSGNTASLTISGL |
| | CARSAYGDYFLDYWGQGTLVTVSS SEQ ID | QAEDEADYYCSSFTSSSTLVVFGGGT |
| | NO. 15 | KVTVL SEQ ID NO. 16 |
| H6 | EVQLLESGGGLVQPGGSLRLSCAASGFTFSS | AIRMTQSPAFMSATPGDKVNISYKASQ |
| | YEMNWVRQAPGKGLEWVSYISSSGSTIYYAD | DVDDDMTWCQEKPGEAAIFIFQEAATL |
| | SVKGRFTISRDNAKNSLYLQMNSLRAEDTAVY | VPGIPPRLSGSGNGTDFTLTINNMESE |
| | YCARDGAATGDQIDYWGQGTLVTVSS SEQ | DAAYYFCLQQDNFPLTFGQGTKVDIK |
| | ID NO. 17 | SEQ ID NO. 18 |
| H8 | EVQLVQSGAEVKKPGASVKVSCKASGYTFSS | QLVLTQSPSVSVAPGQRVTISCSGSNS |
| | YYMHWVRQAPGQGLEWMGWINPNSGNTGY | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDYWGQGTLVTVSS SEQ | GDEAHYYCGTWDSTLSAGVFGGGTKL |
| | ID NO. 19 | TVL SEQ ID NO. 20 |
| H8-9 | EVQLVQSGAEVKKPGASVKVSCKASGYTFYS | QLVLTQSPSVSVAPGQRVTISCSGSNS |
| | YYMHWVRQAPGQGLEWMGWINPNSGNTGY | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 21 | LTVL SEQ ID NO. 22 |
| H8-9EE8L3 | EVQLVQSGAEVKKPGASVKVSCKASGYTFYS | QLVLTQSPSVSVAPGQRVTISCSGSNS |
| | YYMHWVRQAPGQGLEWMGWINPNSGNTGY | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWLFGGGTKL |
| | ID NO. 21 | TVL SEQ ID NO. 23 |
| H8-G35 | EVQLVQSGAEVKKPGASVKVSCKASGYTFYS | QLVLTQSPSVSVAPGQRVTISCSGSNS |
| | EYMHWVRQAPGQGLEWMGWINPNSGNTGY | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 24 | LTVL SEQ ID NO. 22 |
| H8-A2 | EVQLVQSGAEVKKPGASVKVSCKASGYTFYS | QLVLTQSPSVSVAPGQRVTISCSGSNS |
| | EYMHWVRQAPGQGLEWMGWINPNSGNTGV | NIGNNYVSWYHHLPGTAPKLLIYDNNK |
| | APKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWAFGGGTK |
| | ID NO. 25 | LTVL SEQ ID NO. 26 |
| H8-B6 | EVQLVQSGAEVKKPGASVKVSCKASGYTFYS | QLVLTQSPSVSVAPGQRVTISCSGSNS |
| | EYMHWVRQAPGQGLEWMGWINPNSGNTGY | FTDNTYVSWYHHLPGTAPKLLIYDTNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 27 | LTVL SEQ ID NO. 28 |
| H8-C1 | EVQLVQSGAEVKKPGASVKVSCKASGYTFYS | QLVLTQSPSVSVAPGQRVTISCSGSNS |
| | EYMHWVRQAPGQGLEWMGWINPNSGNTGL | NIGNNYVSWYHHLPGTAPKLLIYDNNK |
| | APKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWLFGGGTKL |
| | ID NO. 29 | TVL SEQ ID NO. 23 |
| H8-D4 | EVQLVQSGAEVKKPGASVKVSCKASGYTFYS | QLVLTQSPSVSVAPGQRVTISCSGSNS |
| | EYMHWVRQAPGQGLEWMGWINPNSGNTGY | NIGNNYVSWYHHLPGTAPKLLIYDNNK |
| | AQKFQGRVTMTRNTSISTAYMELSSLRSEDTA | RQSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 24 | LTVL SEQ ID NO. 30 |
| H8-D5 | EVQLVQSGAEVKKPGASVKVSCKASGYTFYS | QLVLTQSPSVSVAPGQRVTISCSGSNS |
| | YYMHWVRQAPGQGLEWMGWINPNSGNTGV | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWLFGGGTKL |
| | ID NO. 31 | TVL SEQ ID NO. 23 |
| H8-D6 | EVQLVQSGAEVKKPGASVKVSCKASGYTFYS | QLVLTQSPSVSVAPGQRVTISCSGSNS |
| | EYMHWVRQAPGQGLEWMGWINPNSGNTGY | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWLFGGGTKL |
| | ID NO. 24 | TVL SEQ ID NO. 23 |
| H8-D10 | EVQLVQSGAEVKKPGASVKVSCKASGYTFYS | QLVLTQSPSVSVAPGQRVTISCSGSNS |
| | EYMHWVRQAPGQGLEWMGWINPNSGNTGL | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWLFGGGTKL |
| | ID NO. 32 | TVL SEQ ID NO. 23 |
| H8-E5 | EVQLVQSGAEVKKPGASVKVSCKASGYTFYS | QLVLTQSPSVSVAPGQRVTISCSGSNS |
| | EYMHWVRQAPGQGLEWMGWINPNSGNTGY | NIGNNYVSWYHHLPGTAPKLLIYDNNK |
| | APKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 33 | LTVL SEQ ID NO. 22 |
| H8-G7 | EVQLVQSGAEVKKPGASVKVSCKASGYTFYS | QLVLTQSPSVSVAPGQRVTISCSGSNS |
| | EYMHWVRQAPGQGLEWMGWINPNSGNTGV | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 34 | LTVL SEQ ID NO. 22 |
| H8-G9 | EVQLVQSGAEVKKPGASVKVSCKASGYTFYS | QLVLTQSPSVSVAPGQRVTISCSGSNS |
| | EYMHWVRQAPGQGLEWMGWINPNSGNTGY | FSSNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 24 | LTVL SEQ ID NO. 35 |
| H8-H6 | EVQLVQSGAEVKKPGASVKVSCKASGYTFYS | QLVLTQSPSVSVAPGQRVTISCSGSNS |
| | YYMHWVRQAPGQGLEWMGWINPNSGNTGL | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWAFGGGTK |
| | ID NO. 36 | LTVL SEQ ID NO. 26 |
| H8-2A2 | EVQLVQSGAEVKKPGASVKVSCKASGYTFYS | QLVLTQSPSVSVAPGQRVTISCSGSNS |
| | EYMHWVRQAPGQGLEWMGWINPNSGNTGL | NIGNNYVSWYHHLPGTAPKLLIYDNNK |
| | APKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 29 | LTVL SEQ ID NO. 22 |
| H8-261 | EVQLVQSGAEVKKPGASVKVSCKASGYTFYS | QLVLTQSPSVSVAPGQRVTISCSGSNS |
| | YYMHWVRQAPGQGLEWMGWINPNSGNTGL | NIGNNYVSWYHHLPGTAPKLLIYDTNK |
| | APKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWAFGGGTK |
| | ID NO. 37 | LTVL SEQ ID NO. 38 |
| H8-262 | EVQLVQSGAEVKKPGASVKVSCKASGYTFYS | QLVLTQSPSVSVAPGQRVTISCSGSNS |
| | EYMHWVRQAPGQGLEWMGWINPNSGNTGV | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWLFGGGTKL |
| | ID NO. 34 | TVL SEQ ID NO. 23 |
| H8-264 | EVQLVQSGAEVKKPGASVKVSCKASGYTFYS | QLVLTQSPSVSVAPGQRVTISCSGSNS |
| | YYMHWVRQAPGQGLEWMGWINPNSGNTGL | NIGNNYVSWYHHLPGTAPKLLIYDNNK |
| | APKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWLFGGGTKL |
| | ID NO. 37 | TVL SEQ ID NO. 23 |
| H8-267 | EVQLVQSGAEVKKPGASVKVSCKASGYTFYS | QLVLTQSPSVSVAPGQRVTISCSASNS |
| | EYMHWVRQAPGQGLEWMGWINPNSGNTGL | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 32 | LTVL SEQ ID NO. 39 |
| H8-A7P | EVQLVQSGAEVKKPGASVKVSCKASGYTFYS | QLVLTQSPSVSVAPGQRVTISCSGSNS |
| | EYMHWVRQAPGQGLEWMGWINPNSGNTGL | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 32 | LTVL SEQ ID NO. 22 |
| GCE-A10 | QVQLVQSGAEVKKPGASVKVSCKASGYTFSG | QSVVTQPPSVSGAPGQRVTISCLGSAS |
| | DYMHWVRQAPGQGLEWMGWINPNSGGTNY | NIGAGHDVHWYQQLPGTAPKLLIYGNS |
| | AQKFQGRVTMTRDTSISTAYMELSRLRSDDT | NRPSGVPDRFSGSKSGTSASLAITGLQ |
| | AVYYCAREPARDYYYYDGLDVWGQGTTVTV | AEDEADYYCQSYSSSLSAYVFGTGTK |
| | SS SEQ ID NO. 40 | VTVL SEQ ID NO. 41 |
| GCE-A11 | QVQLVQSGAEVKKPGASVKVSCKASGFTFSG | QSVVTQPPSVSGAPGQRVTISCLGSSS |
| | DYIHWVRQAPGQGLEWMGWINPNSGGTNYA | NIGAGHDVHWYQQLPGTAPKLLIYGNS |
| | QKFQGRVTMTRDTSISTAYMELSRLRSDDTA | NRISGVPDRFSGSKSGTSASLAITGLQ |
| | VYYCAREPARDYYYYDGLDVWGQGTTVTVS | AEDEADYYCQSYSSSLSAYLFGTGTKV |
| | S SEQ ID NO. 42 | TVL SEQ ID NO. 43 |
| GCE-A13 | QVQLVQSGAEVKKPGASVKVSCKASGYTFSG | QSVVTQPPSVSGAPGQRVTISCLGSAS |
| | DYLHWVRQAPGQGLEWMGWINPNSGGTNY | NIGAGHDVHWYQQLPGTAPKLLIYGNS |
| | AQKFQGRVTMTRDTSISTAYMELSRLRSDDT | NRPSGVPDRFSGSKSGTSASLAITGLQ |
| | AVYYCAREPARDYYYYDGLDVWGQGTTVTV | AEDEADYYCQSYSSSLSAYVFGTGTK |
| | SS SEQ ID NO. 44 | VTVL SEQ ID NO. 41 |
| GCE-A14 | QVQLVQSGAEVKKPGASVKVSCKASGYTFSG | QSVVTQPPSVSGAPGQRVTISCIGSSS |
| | DYIHWVRQAPGQGLEWMGWINPNSGGTNYA | NIGAGHDVHWYQQLPGTAPKLLIYGNS |
| | QKFQGRVTMTRDTSISTAYMELSRLRSDDTA | NRPSGVPDRFSGSKSGTSASLAITGLQ |
| | VYYCAREPARDYYYYDGLDVWGQGTTVTVS | AEDEADYYCQSYSSSLSAYVFGTGTK |
| | S SEQ ID NO. 45 | VTVL SEQ ID NO. 46 |
| GCE-A16 | QVQLVQSGAEVKKPGASVKVSCKASGYTFSG | QSVVTQPPSVSGAPGQRVTISCIGSSS |
| | DYLHWVRQAPGQGLEWMGWINPNTGGTNYA | NIGAGYDVHWYQQLPGTAPKLLIYGNS |
| | QKFQGRVTMTRDTSISTAYMELSRLRSDDTA | NLPSGVPDRFSGSKSGTSASLAITGLQ |
| | VYYCAREPARDYYYYDGLDVWGQGTTVTVS | AEDEADYYCQSYESSLSAYVFGTGTK |
| | S SEQ ID NO. 47 | VTVL SEQ ID NO. 48 |
| GCE-A18 | QVQLVQSGAEVKKPGASVKVSCKASGYTFSG | QSVVTQPPSVSGAPGQRVTISCIGSAS |
| | DYMHWVRQAPGQGLEWMGWINPNSGGTNY | NIGAGHDVHWYQQLPGTAPKLLIYGNS |
| | AQKFQGRVTMTRDTSISTAYMELSRLRSDDT | NRPSGVPDR FSGSKSGTSASLAITGLQ |
| | AVYYCAREPGRDYYYYDGLDVWGQGTTVTV | AEDEADYYCQSYSSSLSAYVFGTGTK |
| | SS SEQ ID NO. 49 | VTVL SEQ ID NO. 50 |
| GCE-B2 | QVQLVQSGAEVKKPGASVKVSCKASGYTFSG | QSVVTQPPSVSGAPGQRVTISCLGSAS |
| | DYMHWVRQAPGQGLEWMGWINPNTGGTNY | NIGAGYDVHWYQQLPGTAPKLLIYGNS |
| | AQKFQGRVTMTRDTSISTAYMELSRLRSDDT | NRPSGVPDRFSGSKSGTSASLAITGLQ |
| | AVYYCAREPARDYYYYDGLDVWGQGTTVTV | AEDEADYYCQSYSSSLSAYVFGTGTK |
| | SS SEQ ID NO. 51 | VTVL SEQ ID NO. 52 |
| GCE-B9 | QVQLVQSGAEVKKPGASVKVSCKASGFTFSG | QSVVTQPPSVSGAPGQRVTISCLGSSS |
| | DYMHWVRQAPGQGLEWMGWINPNSGGTNY | NIGAGHDVHWYQQLPGTAPKLLIYGNS |
| | AQKFQGRVTMTRDTSISTAYMELSRLRSDDT | NLPSGVPDRFSGSKSGTSASLAITGLQ |
| | AVYYCAREPARDYYYYDGLDVWGQGTTVTV | AEDEADYYCQSYSSSLSAVLFGTGTKV |
| | SS SEQ ID NO. 53 | TVL SEQ ID NO. 54 |
| GCE-B11 | QVQLVQSGAEVKKPGASVKVSCKASGYTFSG | QSVVTQPPSVSGAPGQRVTISCIGSSS |
| | DYIHWVRQAPGQGLEWMGWINPNSGGTNYA | NIGAGYDVHWYQQLPGTAPKLLIYGNS |
| | QKFQGRVTMTRDTSISTAYMELSRLRSDDTA | NRISGVPDRFSGSKSGTSASLAITGLQ |
| | VYYCAREPARDYYYYDGLDVWGQGTTVTVS | AEDEADYYCQSYSSSLSAVLFGTGTKV |
| | S SEQ ID NO. 45 | TVL SEQ ID NO. 55 |
| GCE-B13 | QVQLVQSGAEVKKPGASVKVSCKASGSTFSG | QSVVTQPPSVSGAPGQRVTISCLGSAS |
| | DYIHWVRQAPGQGLEWMGWINPNSGGTNYA | NIGAGHDVHWYQQLPGTAPKLLIYGNS |
| | QKFQGRVTMTRDTSISTAYMELSRLRSDDTA | NRPSGVPDRFSGSKSGTSASLAITGLQ |
| | VYYCAREPARDYYYYDGLDVWGQGTTVTVS | AEDEADYYCQSYSSSLSAVLFGTGTKV |
| | S SEQ ID NO. 56 | TVL SEQ ID NO.57 |
| GCE-B19 | QVQLVQSGAEVKKPGASVKVSCKASGFTFSG | QSVVTQPPSVSGAPGQRVTISCLGSAS |
| | DYIHWVRQAPGQGLEWMGWINPNSGGTNYA | NIGAGHDVHWYQQLPGTAPKLLIYGNS |
| | QKFQGRVTMTRDTSISTAYMELSRLRSDDTA | NRPSGVPDRFSGSKSGTSASLAITGLQ |
| | VYYCAREPGRDYYYYDGLDVWGQGTTVTVS | AEDEADYYCQSYSSSLSAVLFGTGTKV |
| | S SEQ ID NO. 58 | TVL SEQ ID NO. 57 |
| GCE-BR1 | QVQLVQSGAEVKKPGASVKVSCKASGSTFSG | QSVVTQPPSVSGAPGQRVTISCLGSAS |
| | DYLHWVRQAPGQGLEWMGWINPNSGGTNY | NIGAGYDVHWYQQLPGTAPKLLIYGNS |
| | AQKFQGRVTMTRDTSISTAYMELSRLRSDDT | NRPSGVPDRFSGSKSGTSASLAITGLQ |
| | AVYYCAREPARDYYYYDGLDVWGQGTTVTV | AEDEADYYCQSYSSSLSAVLFGTGTKV |
| | SS SEQ ID NO. 59 | TVL SEQ ID NO. 60 |
| GCE-B20 | QVQLVQSGAEVKKPGASVKVSCKASGYTFSG | QSVVTQPPSVSGAPGQRVTISCLGSAS |
| | DYLHWVRQAPGQGLEWMGWINPNSGGTNY | NIGAGHDVHWYQQLPGTAPKLLIYGNS |
| | AQKFQGRVTMTRDTSISTAYMELSRLRSDDT | NRISGVPDRFSGSKSGTSASLAITGLQ |
| | AVYYCAREPARDYYYYDGMDVWGQGTTVTV | AEDEADYYCQSYSSSLSAVLFGTGTKV |
| | SS SEQ ID NO. 61 | TVL SEQ ID NO. 62 |
| GCE-A19 | QVQLVQSGAEVKKPGASVKVSCKASGFTFSG | QSVVTQPPSVSGAPGQRVTISCLGSSS |
| | DYLHWVRQAPGQGLEWMGWINPNSGGTNY | NIGAGHDVHWYQQLPGTAPKLLIYGNS |
| | AQKFQGRVTMTRDTSISTAYMELSRLRSDDT | NRISGVPDRFSGSKSGTSASLAITGLQ |
| | AVYYCAREPARDYYYYYGLDVWGQGTTVTVS | AEDEADYYCQSYSSSLSAYVFGTGTK |
| | S SEQ ID NO. 63 | VTVL SEQ ID NO. 64 |
| GCE-B10 | QVQLVQSGAEVKKPGASVKVSCKASGFTFSG | QSVVTQPPSVSGAPGQRVTISCLGSSS |
| | DYLHWVRQAPGQGLEWMGWINPNTGGTNYA | NIGAGHDVHWYQQLPGTAPKLLIYGNS |
| | QKFQGRVTMTRDTSISTAYMELSRLKSDDTAV | NLPSGVPDRFSGSKSGTSASLAITGLQ |
| | YYCAREPARDYYYYDGLDVWGQGTTVTVSS | AEDEADYYCQSYSSSLSAYVFGTGTK |
| | SEQ ID NO. 65 | VTVL SEQ ID NO. 66 |
| GCE-B5 | QVQLVQSGAEVKKPGASVKVSCKASGFTFSG | QSVVTQPPSVSGAPGQRVTISCLGSAS |
| | DYIHWVRQAPGQGLEWMGWINPNSGGTNYA | NIGAGHDVHWYQQLPGTAPKLLIYGNS |
| | QKFQGRVTMTRDTSISTAYMELSRLRSDDTA | NRPSGVPDRFSGSKSGTSASLAITGLQ |
| | VYYCAREPGRDYYYYDGLDVWGQGTTVTVS | AEDEADYYCQSYSSSLSAVVFGTGTK |
| | S SEQ ID NO. 58 | VTVL SEQ ID NO. 67 |
| GCE-B4 | QVQLVQSGAEVKKPGASVKVSCKASGYTFSG | QSVVTQPPSVSGAPGQRVTISCIGSAS |
| | DYLHWVRQAPGQGLEWMGWINPNSGGTNY | NIGAGHDVHWYQQLPGTAPKLLIYGNS |
| | AQKFQGRVTMTRDTSISTAYMELSRLRSDDT | NRISGVPDRFSGSKSGTSASLAITGLQ |
| | AVYYCAREPARDYYYYDGMDVWGQGTTVTV | AEDEADYYCQSYSSSLSAVLFGTGTKV |
| | SS SEQ ID NO. 61 | TVL SEQ ID NO. 68 |
| GCE-A26 | QVQLVQSGAEVKKPGASVKVSCKASGFTFSG | QSVVTQPPSVSGAPGQRVTISCLGSSS |
| | DYLHWVRQAPGQGLEWMGWINPNSGGTNY | NIGAGHDVHWYQQLPGTAPKLLIYGNS |
| | AQKFQGRVTMTRDTSISTAYMELSRLRSDDT | NRPSGVPDRFSGSKSGTSASLAITGLQ |
| | AVYYCAREPARDYYYYDGLDVWGQGTTVTV | AEDEADYYCQSYSSSLSAYLFGTGTKV |
| | SS SEQ ID NO. 69 | TVL SEQ ID NO. 70 |
| GCE-L1A-9 | QVQLVQSGAEVKKPGASVKVSCKASGYTFSG | QSVVTQPPSVSGAPGQRVTISCLGSSS |
| | DYMHWVRQAPGQGLEWMGWINPNSGGTNY | NIGAGYDVHWYQQLPGTAPKLLIYGNS |
| | AQKFQGRVTMTRDTSISTAYMELSRLRSDDT | NRPSGVPDRFSGSKSGTSASLAITGLQ |
| | AVYYCAREPGRDYYYYDGMDVWGQGTTVTV | AEDEADYYCQSYSSSLSAYVFGTGTK |
| | SS SEQ ID NO. 71 | VTVL SEQ ID NO. 72 |
| GCE-H3B-36 | QVQLVQSGAEVKKPGASVKVSCKASGYTFSG | QSVVTQPPSVSGAPGQRVTISCTGSS |
| | DYMHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPGRDYYYYDGLDVWGQGTTVTV | QAEDEADYYCQSYSSSLSAYVFGTGT |
| | SS SEQ ID NO. 49 | KVTVL SEQ ID NO.73 |
| GCE-H13-1 | QVQLVQSGAEVKKPGASVKVSCKASGSTFSG | QSVVTQPPSVSGAPGQRVTISCTGSS |
| | DYLHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPGRDYYYYDGLDVWGQGTTVTV | QAEDEADYYCQSYSSSLSAYVFGTGT |
| | SS SEQ ID NO. 74 | KVTVL SEQ ID NO. 73 |
| GCE-H13-2 | QVQLVQSGAEVKKPGASVKVSCKASGYTFSG | QSVVTQPPSVSGAPGQRVTISCTGSS |
| | DYLHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPARDYYYYDGMDVWGQGTTVTV | QAEDEADYYCQSYSSSLSAYVFGTGT |
| | SS SEQ ID NO. 61 | KVTVL SEQ ID NO. 73 |
| GCE-H13-3 | QVQLVQSGAEVKKPGASVKVSCKASGYTFSG | QSVVTQPPSVSGAPGQRVTISCTGSS |
| | DYLHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPARDYYYYDGLDVWGQGTTVTV | QAEDEADYYCQSYSSSLSAYVFGTGT |
| | SS SEQ ID NO. 44 | KVTVL SEQ ID NO. 73 |
| GCE-H13-4 | QVQLVQSGAEVKKPGASVKVSCKASGYTFSG | QSVVTQPPSVSGAPGQRVTISCTGSS |
| | DYMHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPARDYYYYDGLDVWGQGTTVTV | QAEDEADYYCQSYSSSLSAYVFGTGT |
| | SS SEQ ID NO. 40 | KVTVL SEQ ID NO. 73 |
| GCE-H13-5 | QVQLVQSGAEVKKPGASVKVSCKASGFTFSG | QSVVTQPPSVSGAPGQRVTISCTGSS |
| | DYLHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPARDYYYYDGMDVWGQGTTVTV | QAEDEADYYCQSYSSSLSAYVFGTGT |
| | SS SEQ ID NO. 75 | KVTVL SEQ ID NO. 73 |
| GCE-H13-6 | QVQLVQSGAEVKKPGASVKVSCKASGFTFSG | QSVVTQPPSVSGAPGQRVTISCTGSS |
| | DYLHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPARDYYYYDGLDVWGQGTTVTV | QAEDEADYYCQSYSSSLSAYVFGTGT |
| | SS SEQ ID NO. 69 | KVTVL SEQ ID NO. 73 |
| GCE-H13-8 | QVQLVQSGAEVKKPGASVKVSCKASGYTFSG | QSVVTQPPSVSGAPGQRVTISCTGSS |
| | DYLHWVRQAPGQGLEWMGWINPNSGGTNY | SNIGAGYDVHWYQQLPGTAPKLLIYGN |
| | AQKFQGRVTMTRDTSISTAYMELSRLRSDDT | SNRPSGVPDRFSGSKSGTSASLAITGL |
| | AVYYCAREPGRDYYYYDGLDVWGQGTTVTV | QAEDEADYYCQSYSSSLSAYVFGTGT |
| | SS SEQ ID NO. 76 | KVTVL SEQ ID NO. 73 |
| H8-9EH11L | EVQLVQSGAEVKKPGASVKVSCKASGYTFYS | QLVLTQSPSVSVAPGQRVTISCSGSNS |
| | YYMHWVRQAPGQGLEWMGWINPNSGNTGY | FIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 21 | LTVL SEQ ID NO. 77 |
| H8-9EG11L | EVQLVQSGAEVKKPGASVKVSCKASGYTFYS | QLVLTQSPSVSVAPGQRVTISCSGSNS |
| | YYMHWVRQAPGQGLEWMGWINPNSGNTGY | NIGNTYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRGTTVSFDTWGQGTLVTVSS SEQ | GDEAHYYCGTWDSTLSAWVFGGGTK |
| | ID NO. 21 | LTVL SEQ ID NO. 78 |
| H8-6AG2H3 | EVQLVQSGAEVKKPGASVKVSCKASGYTFSD | QLVLTQSPSVSVAPGQRVTISCSGSNS |
| | YYMHWVRQAPGQGLEWMGWINPNSGNTGY | NIGNNYVSWYHHLPGTAPKLLIYDNNK |
| | AQKFQGRVTMTRNTSISTAYMELSSLRSEDTA | RPSGIPDRFSGSKSGTSATLGITGLQP |
| | VYYCARRATTVSFDYWGQGTLVTVSS SEQ | GDEAHYYCGTWDSTLSAGVFGGGTKL |
| | ID NO. 79 | TVL SEQ ID NO. 20 |
| A1-2 | QVQLQESGPGLVKPSQTLSLTCTVSGGSISSG | LPVLTQPASVSGSPGQSITISCTGTSFD |
| | GYYWSWIRQHPGKGLEWIGESTHSGSTNYN | VGGYNYVSWYQQHPGKAPKLMIYDVS |
| | PSLKSRVTISVDTSKNQFSLKLSSVTAADTAVY | DRPSGVSTRFSGSKSGNTASLTISGLQ |
| | YCARGRDGYDFDAWGQGTLVTVSS SEQ ID | AEDEADYYCSSFRSSSALVVFGGGTKL |
| | NO. 80 | TVL SEQ ID NO. 81 |
| A1-4 | QVQLQESGPGLVKPSQTLSLTCTVSGGSISSG | LPVLTQPASVSGSPGQSITISCTGTSSD |
| | GYYWSWIRQHPGKGLEWIGESSHSGSTNYN | VGGYPYVSWYQQHPGKAPKLMIYVVS |
| | PSLKSRVTISVDTSKNQFSLKLSSVTAADTAVY | DRPSGVSTRFSGSKSGNTASLTISGLQ |
| | YCARGRDGYYFDAWGQGTLVTVSS SEQ ID | AEDEADYYCSSYRSSSALVVFGGGTQ |
| | NO. 82 | LTVL SEQ ID NO. 83 |
| A1-6 | QVQLQESGPGLVKPSQTLSLTCTVSGGSISSG | LPVLTQPASVSGSPGQSITISCTGTSW |
| | GYYWSWIRQHPGKGLEWIGEITHSGSTNYNP | DVGGYPYVSWYQQHPGKAPKLMIYDV |
| | SLKSRVTISVDTSKNQFSLKLSSVTAADTAVYY | SDRPSGVSTRFSGSKSGNTASLTISGL |
| | CARGRDGYDIDAWGQGTLVTVSS SEQ ID | QAEDEADYYCSSYRSVSALVVFGGGT |
| | NO. 84 | KLTVL SEQ ID NO. 85 |
| A1-8 | QVQLQESGPGLVKPSQTLSLTCTVSGGSISSG | LPVLTQPASVSGSPGQSITISCTGTSSD |
| | GYYWSWIRQHPGKGLEWIGEISHSGSTNYNP | VGGYPYVSWYQQHPGKAPKLMIYRVS |
| | SLESRVTISVDTSKNQFSLKLSSVTAADTAVYY | DRPSGVSTRFSGSKSGNTASLTISGLQ |
| | CARGRDGYDLDRWGQGTLVTVSS SEQ ID | AEDEADYYCSSYRSSAALVVFGGGTK |
| | NO. 86 | LTVL SEQ ID NO. 87 |
| A1-9 | QVQLQESGPGLVKPSQTLSLTCTVSGGSISSG | LPVLTQPASVSGSPGQSITISCTGTSSD |
| | GYYWSWIRQHPGKGLEWIGEISHSGSTNYNP | VGGYNYVSWYQQHPGKAPKLMIYNVS |
| | SLKSRVTISVDTSKNQFSLKLSSVTAADTAVYY | DRPSGVSTRFSGSKSGNTASLTISGLQ |
| | CARGRDGYYLDQWGQGTLVTVSS SEQ ID | AEDEADYYCSSFRSSSALVVFGGGTKL |
| | NO. 88 | TVL SEQ ID NO. 89 |
| A1-24 | QVQLQESGPGLVKPSQTLSLTCTVSGGSISSG | LPVLTQPASVSGSPGQSITISCTGTSFD |
| | GYYWSWIRQHPGKGLEWIGESTHSGSTNYN | VGGYNYVSWYQQHPGKAPKLMIYDVS |
| | PSLESRVTISVDTSKNQFSLKLSSVTAADTAVY | DRPSGVSTRFSGSKSGNTASLTISGLQ |
| | YCARGRDSYDFDAWGQGTLVTVSS SEQ ID | AEDEADYYCSSFRSSAALVVFGGGTKL |
| | NO. 90 | TVL SEQ ID NO. 91 |
| A1-32 | QVQLQESGPGLVKPSQTLSLTCTVSGGSISSG | LPVLTQPASVSGSPGQSITISCTGTSFD |
| | GYYWSWIRQHPGKGLEWIGESTHSGSTNYN | VGGYPYVSWYQQHPGKAPKLMIYDVS |
| | PSLDSRVTISVDTSKNQFSLKLSSVTAADTAVY | DRPSGVSTRFSGSKSGNTASLTISGLQ |
| | YCARGRDGYYLDQWGQGTLVTVSS SEQ ID | AEDEADYYCSSFRSSAALVVFGGGTKL |
| | NO. 92 | TVL SEQ ID NO. 93 |

We claim:

1. An antibody drug conjugate (ADC) composition comprising an IgG antibody that binds to c-Met, a conjugation linker moiety that binds to both Cys residues in the hinge region of an IgG antibody and a toxin moiety.

2. The ADC composition of claim 1, wherein the toxin moiety is a tubulin inhibitor or a doxorubicin analog.

3. The ADC composition of claim 1, wherein the antibody is an IgG antibody selected from H8, B12, H8-A2, H8-9, H8-9EE8L3, H8-C1, H8-D4, H8-D5, H8-D6, H8-D10, H8-E5, H8-G7, H8-H6, H8-2A2, H8-2B1, H8-2B2, H8-2B4, H8-2B7, H8-A7P, H8-9EH11L, H8-9EH11L, H8-9EGH11L, and H8-6AG2H3.

4. The ADC composition of claim 1, wherein the conjugated toxin is

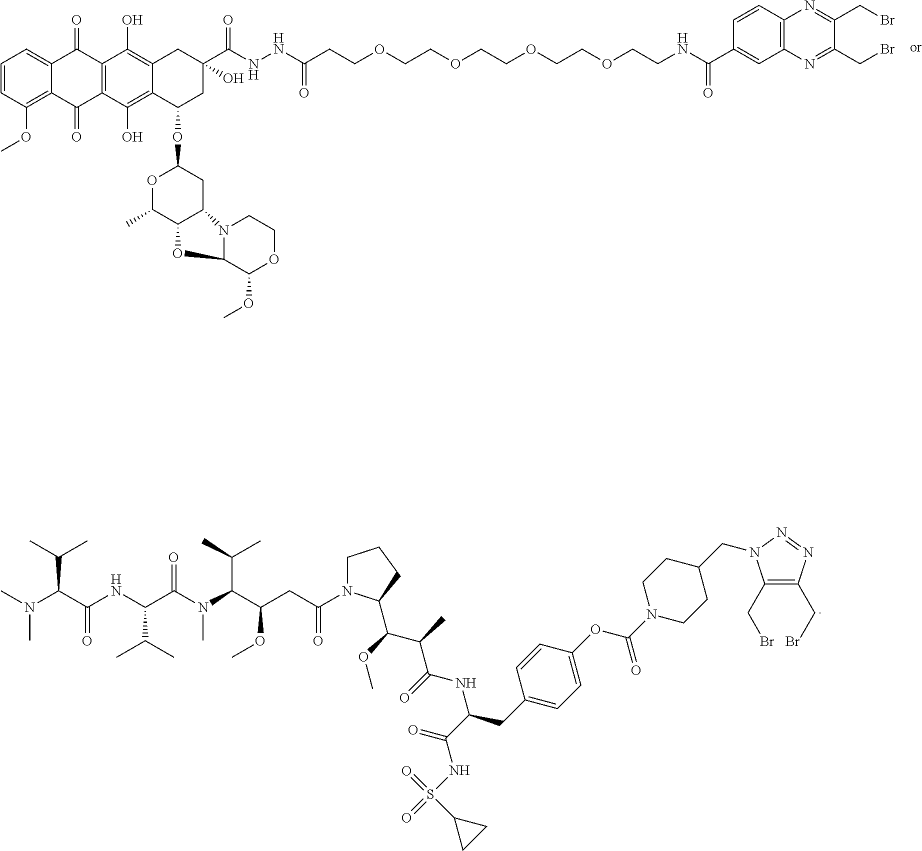

5. A method for treating breast cancer, comprising administering an effective amount of an antibody drug conjugate (ADC) composition comprising an IgG antibody that binds to c-Met, a conjugation linker moiety that binds to both Cys residues in the hinge region of an IgG antibody and a toxin moiety.

6. The method for treating breast cancer of claim 5, wherein the toxin moiety is a tubulin inhibitor or a doxorubicin analog.

7. The method for treating breast cancer of claim 5, wherein the antibody is an IgG antibody selected from H8, B12, H8-A2, H8-9, H8-9EE8L3, H8-C1, H8-D4, H8-D5, H8-D6, H8-D10, H8-E5, H8-G7, H8-H6, H8-2A2, H8-2B1, H8-2B2, H8-2B4, H8-2B7, H8-A7P, H8-9EH11L, H8-9EGH11L, and H8-6AG2H3.

8. The method for treating breast cancer of claim 5, wherein the conjugated toxin is 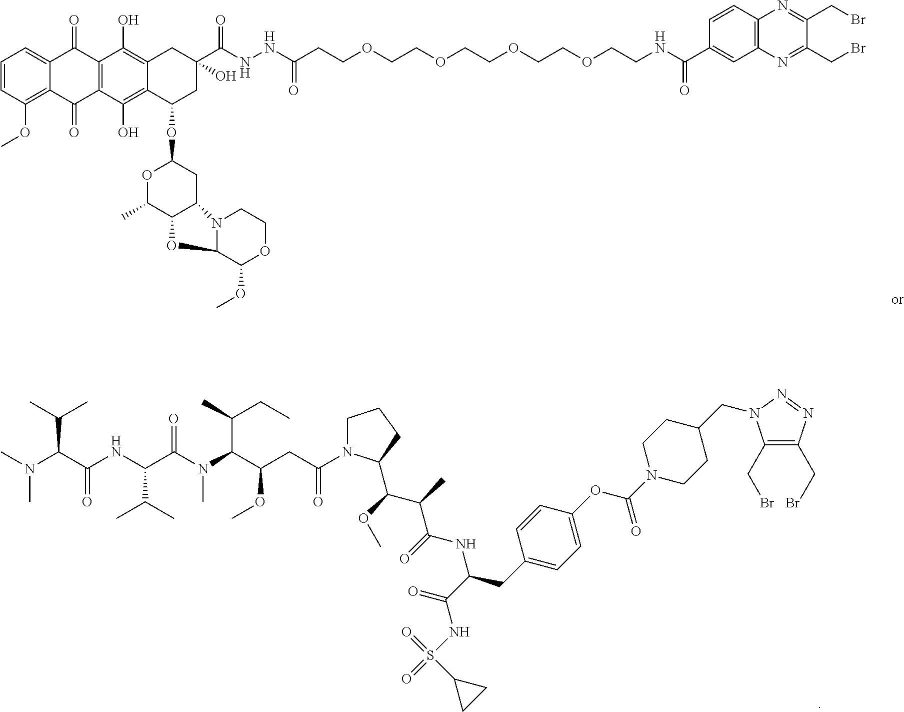 [SMILES: COc1cccc2c1C(=O)c1c(O)c3c(c(O)c1C2=O)C[C@@](O)(C(=O)NNC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1 ; CC[C@H](C)[C@@H]([C@@H](CC(=O)N1CCC[C@H]1[C@H](OC)[C@@H](C)C(=O)N[C@@H](Cc1ccc(OC(=O)N2CCC(Cn3nnc(CBr)c3CBr)CC2)cc1)C(=O)NS(=O)(=O)C1CC1)OC)N(C)C(=O)[C@@H](NC(=O)[C@H](C(C)C)N(C)C)C(C)C]
